(19) United States
(12) Patent Application Publication
(10) Pub. No.: US 2006/0160702 A1
(43) Pub. Date: Jul. 20, 2006

(54) Compositions comprising anti-proliferative agents and use thereof

(76) Inventors: Etienne Soudant, Paris (FR); Lea Von Oppen-Bezalel, Berlin (DE); Meira Ziv, Rehovot (IL); Inon Perry, Tel Aviv (IL)

Correspondence Address: FENNEMORE CRAIG, 3003 NORTH CENTRAL AVENUE, SUITE 2600, PHOENIX, AZ, 85012, US

(21) Appl. No.: 11/289,156

(22) Filed: Nov. 28, 2005

Related U.S. Application Data
(63) Continuation-in-part of application No. 10/465,911, filed on Jun. 20, 2003.
Application No. 10/465,911 is a continuation of application No. 09/915,768, filed on Jul. 27, 2001, now Pat. No. 6,635,287.
Application No. 09/915,768 is a continuation of application No. 09/367,898, filed on Nov. 29, 1999, now Pat. No. 6,342,254.

(51) Int. Cl. A01N 63/00; A61K 36/18

(52) U.S. Cl. 504/117; 424/725

(57) ABSTRACT

The invention relates to anti-proliferative agents derived from plants, wherein the agents are capable of inducing a plant organ into a state of dormancy or maintaining the organ in the state of dormancy. The invention further discloses compositions comprising the anti-proliferative agents and the use of said compositions to inhibit undesired or deleterious cell proliferation in plant or mammal tissue.

This application is a continuation-in-part of U.S. patent application Ser. No. 10/465,911 filed Jun. 20, 2003, which is a continuation of U.S. patent application Ser. No. 09/915,768, now U.S. Pat. No. 6,635,287, filed Jul. 27, 2001, which is a continuation of U.S. patent application Ser. No. 09/367,898 now U.S. Pat. No. 6,342,254, filed Nov. 29, 1999 as a 371 International Application PCT/IL98/00085 filed Feb. 23, 1998, the entire contents of each of which are hereby incorporated by reference in their entirety.

### FIELD OF THE INVENTION

The present invention relates to compositions comprising plant-derived anti-proliferative agents capable of inducing a plant organ into a state of dormancy or maintaining the organ in the state of dormancy, and the use of said compositions to inhibit undesired or deleterious cell proliferation in plant or mammal tissue.

### BACKGROUND OF THE INVENTION

The term “dormancy” is frequently used in association with plants as well as with animals. However, the definition of this phenomenon is still ambiguous. This uncertainty may be due to the different ways in which dormancy is induced, maintained and broken in different species, and to different states of dormancy which may exist among organs of the same species. Dormancy is widespread in the plant kingdom, and examples can be found in seeds, apical and lateral vegetative buds, floral buds, bulbs, corms and tubers.

In all forms of dormancy, the development of new plant organs from a meristemic tissue is arrested. Therefore, dormancy may be generally defined as the temporary suspension of the growth of meristemic structures, even though the environmental conditions may be favorable for growth.

One of the most studied models of dormancy is seed dormancy. Seeds are the primary dispersal units of higher plants containing the complete genetic information of the species. Seeds are complex biological structures, which, over millions of years, have adapted to divers and often harsh environmental conditions. Seeds are generally able to withstand drought and extreme conditions and may remain viable for prolonged periods of time, which can extend to hundred of years. Seeds consist of nutrient reserve storage tissue(s) (endosperm or perisperm), embryo, and encapsulating structure that protects the embryo and may also participate in the regulation of germination (fruit or dispersal organ).

A common misconception is that seed dormancy simply means that a seed has not germinated; however, this definition is utterly inadequate. Unfavorable environmental conditions are one reason for lack of seed germination. That is, seed could be in a paper bag on the laboratory shelf (i.e. lack of water), buried in a mud in the bottom of a lake (i.e. lack of oxygen and/or light) or exposed to temperatures that are above or below those suitable for plant growth. Such non-germinating seeds may be non-dormant or dormant. A non-dormant seed will germinate under favorable conditions, whereas a dormant seed will usually display much greater restrictions in terms of the conditions required for it to germinate.

During maturation seeds may enter a state of true primary dormancy, which may or may not be sustained after maturity. Before germination can occur in mature, dormant seeds, a set of conditions must be fulfilled in order to break their dormancy. The requirements for dormancy relief may be different from those for germination. A more accurate definition for seed dormancy may therefore be the inability of seeds to germinate under favorable environmental conditions. This definition is also correct for other plant dispersal organs such as corms, bulbs and tubers.

According to Nikolaeva (Nikolaeva, M. G. 1969. Physiology of deep dormancy in seeds. Izdatel'stvo “Nauka” Leningrad (Translated from Russian by Z. Shapiro, National Science Foundation, Washington D.C.); Nikolaeva, M. G. 1977. Factors controlling the seed dormancy and germination. In: The Physiology and Biochemistry of Seed Dormancy and Germination, A. A. Khan, ed., pp. 51-74. North-Holland, Amsterdam/New York), there are two general types of primary seed dormancy: endogenous and exogenous. In endogenous dormancy, some characteristics of the embryo prevent germination, whereas in exogenous dormancy, some characteristic of the surrounding structure covering the embryo, including endosperm (sometime perisperm), seed coat, or fruit structures, prevent germination.

Seed dormancy may be further defined by the following categories: physiological dormancy; morphological dormancy; morphophysiological dormancy; physical dormancy and chemical dormancy (Chapter 3, p. 27-47 In: Seeds, Ecology, Biogeography, and evolution of dormancy and germination. 2001. Baskin C. C. and Baskin J. M. Eds. Academic Press, A Harcourt Science and Technology Company) Physiological dormancy is caused by physiological inhibiting mechanisms within the embryo or its surrounding structures that prevent radicel emergence. In morphological dormancy, the embryo is either non differentiated or underdeveloped. Morphophysiological dormancy is a combination of morphological and physiological dormancy, i.e., the underdeveloped embryo has physiological dormancy. In physical dormancy, the primary reason for the lack of germination is the impermeability of the seeds or its surrounding structures to water. In chemical dormancy, seeds do not germinate under favorable conditions due to the presence of inhibitors that are either produced in or translocated to the seed, where they block embryo growth. These dormancy categories may also define dormancy in other meristemic tissues of plant organs capable of entering into the state of dormancy.

Controlling seed dormancy has an enormous economical implication. Unified release of dormancy from a bulk of seeds leads to uniform germination, which simplifies cultivation and provides better yields. Early breakage of dormancy may give an early, more profitable yield. For example, U.S. Pat. No. 5,912,415 discloses a molecular genetic approach for controlling the expression of gibberellins, plant hormones that control many developmental processes including seed development and germination. U.S. Pat. No. 6,331,504 discloses a method for enhancing spring emergence of fall-seeded crucifers, by exposing the seeds to certain aqueous solutions. U.S. Pat. No. 6,449,899 discloses a method for improved seed germination in a high altitude medicinal plant by exposure to hot water treatment.

On the other hand, sustaining uniform dormancy prevents early sprouting and enables longer storage periods. For example, U.S. Pat. No. 4,247,989 discloses a method for identifying and maintaining a dormancy index in stored grain. U.S. Pat. No. 5,294,593 describes a method to induce dormancy in non-dormant seeds, by employing a set of light and temperature conditions. U.S. Pat. No. 5,635,452 describes the suppression of sprouting in stored potato using aromatic acids.

As explained herein above, a tissue that may enter the state of dormancy is a proliferating tissue, and as dormancy is induced, cell proliferation is arrested. When dormancy is induced by chemical compounds, such compounds may be defined as anti-proliferative agents.

Several plant-derived substances having an effect on cell proliferation have been reported. For example, vinleurosine, vinrosidine, vinblastine and vincristine, alkaloids extracted from the Vinca rosea (Catharanthus roseus), commonly known as the periwinkle plant, possess significant anti-tumor activity. In particular, vinblastine and vincristine have been widely used as single agents and in combination with another antineoplastic drugs in cancer chemotherapy. Another alkaloid, Narciclasine, obtained from bulbs of various Narcissus varieties was shown to inhibit growth of wheat kernel radicels (Ceriotti, G., et al., Tumors 53:359-371 (1967)). Bulbs of Pancratium littoral collected in Hawaii were found to contain a product designated pancratistatin capable of inhibiting growth of various neoplastic cell lines in vitro (Pettit, G. R., et al., J. Nat. Prod, 49:995-1002 (1986)). U.S. Pat. No. 6,489,134 provides novel compounds derived from a marine sponge, Adocia sp. that act as potent anti-mitogens.

However, the cytostatic activity of the above-exemplified compounds is also cytotoxic. Such compound may therefore be used only when cell proliferation should be permanently terminated, and the compounds are directed to the targeted hyperproliferating cells.

Ulex europaeus seed extracts were shown to have non-toxic cytostatic activity, as they reversibly inhibited the growth of certain lymphocytes and various reticuloendothelial tumor cell lines. However, this inhibitory activity was shown only after deliberate stimulation of cell proliferation (Pirofsky, B., et al., Vox-Sang, 42:295-303, (1982) and Pirofsky, B., et al., J. Biol. Response Mod., 2:175-185, (1983)).

Thus, there is a recognized need for, and it would be highly advantageous to have naturally derived, non-toxic anti-proliferative agents for slowing or inhibiting cell proliferation.

### SUMMARY OF THE INVENTION

The present invention relates to a novel approach for slowing cell proliferation, based in part on the phenomenon that specific plant species, in which at least one organ can enter into the state of dormancy, contain compounds that are capable of inducing the state of dormancy or maintaining the state of dormancy in this organ.

As used herein, dormancy is a physiological state wherein the growth of a meristemic tissue is reversibly slowed or ceased.

Compounds that induce or maintain dormancy are therefore defined throughout the present invention as anti-proliferative agents.

It is an object of the present invention to provide compositions comprising at least one plant-derived, water soluble anti-proliferative agent capable of inhibiting proliferation of exogenic cells in a reversible manner.

It is another object of the present invention to provide cosmetic and pharmaceutical compositions comprising as an active ingredient at least one plant-derived, water-soluble anti-proliferative agent, and methods for using same.

It is further object of the present invention to provide agricultural compositions comprising as an active ingredient at least one plant-derived, water-soluble anti-proliferative agent and methods of using same.

According to one aspect, the present invention provides an anti-proliferative composition comprising at least one plant derived, anti-proliferative agent, wherein the anti-proliferative agent:

(a) is a water soluble, small organic molecule;

(b) induces or maintains dormancy in at least one organ of the plant;

(c) inhibits exogenic cell proliferation; and

(d) its inhibitory activity is reversible.

According to certain embodiments, the anti-proliferative composition comprises anti-proliferative agents having an average molecular weight of less than 5,000 Dalton. According to additional embodiments, the anti-proliferative agent is heat stable.

The inhibition of cell proliferation is measured by exposing a tissue or cell culture to different concentrations of the composition and measuring the proliferation rate of the normal tissue or cell culture, wherein a decrease in the proliferation rate as compared to the proliferation rate of the tissue or cell culture incubated without the anti-proliferative composition is observed.

According to one embodiment, the reduction in the proliferation rate of the exogenic cells is at least about 20%, preferably at least about 40%, more preferably at least about 60%, most preferably at least about 80% or more reduction.

The present invention shows that surprisingly, compounds that are capable of inducing dormancy in a plant organ can slow the proliferation of exogenic cells, wherein the exogenic cells may be plant cells or mammalian cells, including human cells.

The anti-proliferative agents according to the present invention can be obtained from any plant organ that produces compounds which are responsible for the entry of a specific plant organ into the state of dormancy, or which maintain such state of dormancy.

According to one embodiment, the anti-proliferative agent according to the present invention is obtained from a dormant plant organ selected from the group consisting of, but not limited to, a seed, an apical and lateral vegetative bud, a floral bud, a bulb, a corm, and a tuber.

According to another embodiment, the anti-proliferative agent according to the present invention is obtained from a plant tissue surrounding a dormant organ or part thereof. According to one embodiment, the dormant organ and the tissue surrounding same compose a plant dispersal organ. According to one currently preferred embodiment, the dormant organ is a seed and the tissue surrounding same is a fruit or part thereof.

Chemical dormancy is not associated with a specific plant family or species. In screening for fruit containing dormancy inducing compounds, fruit in which premature seed sprouting does not occur were first selected. Methods for obtaining anti proliferating agents from such fruit depend on the fruit structure. According to certain embodiments, the anti-proliferative agents within a fruit are obtained by water extraction. According to additional embodiments, the anti-proliferative agents are obtained by separating the aqueous fraction surrounding the seeds within a fleshy fruit. According to one embodiment, the anti-proliferative agents according to the present invention are obtained from a fruit selected from the group consisting of, but not limited to, grape, kiwi, grapefruit, tomato and pitaya.

According to one embodiment, the anti-proliferative composition of the present invention is formulated in a form selected from the group consisting of a solution, a suspension, an emulsion and a dry soluble lyophilized powder. Optionally, the formulation further comprises at least one additional ingredient selected from the group consisting of a preservative and an antioxidant.

According to another aspect, the present invention provides cosmetic and pharmaceutical compositions comprising as an active ingredient an anti-proliferative aqueous composition according to the present invention.

The cosmetic industry is constantly looking for new and improved compounds for skin care, particularly for compounds having antiaging effects. The present invention now discloses that slowing cell proliferation has a beneficial effect in preventing skin aging. Cumulative experimental data have been published favoring the idea that a cell can undergo a definite number of cell divisions. Thus, without wishing to be bound to a specific mechanism, the lower rate of cell proliferation can maintain the cell resources and slow down skin aging.

According to one embodiment, the present invention provides a cosmetic composition comprising as an active ingredient a composition comprising at least one plant derived, anti-proliferative agent, wherein the anti-proliferative agent is a water soluble small organic molecule; induces or maintains dormancy in at least one organ of the plant; inhibits exogenic cell proliferation; and its inhibitory activity is reversible, further comprising a cosmetically acceptable diluent or carrier. According to certain embodiments, the at least one anti-proliferative agent has a molecular weight of less than about 5,000 Dalton. According to additional embodiments, the anti-proliferative agent is heat stable.

According to one embodiment, the cosmetic composition optionally further comprises at least one agent selected from the group consisting of, but not limited to, a preservative, a thickener, a dispersing agent, an emulsifier, a colorant and a perfume, optionally further comprising at least one active ingredient selected from the group consisting of, but not limited to, an anti-oxidant, an anti-inflammatory agent, a moisturizer, a vitamin, a carotenoid, a UV absorbing agent and a UV protecting agent.

Compositions comprising non-toxic anti-proliferative agents have also a significant therapeutic value in the treatment of undesired or deleterious cell proliferation.

Thus, according to yet another aspect, the present invention provides a pharmaceutical composition comprising as an active ingredient a therapeutically effective amount of a composition comprising at least one plant derived, anti-proliferative agent, wherein the anti-proliferative agent is a water soluble, small organic molecule; induces or maintains dormancy in at least one organ of the plant; inhibits exogenic cell proliferation; and its inhibitory activity is reversible, further comprising a diluent, excipient or carrier. According to certain embodiments, the at least one anti-proliferative agent has a molecular weight of less than about 5,000 Dalton. According to additional embodiments, the anti-proliferative agent is heat stable.

According to certain embodiments, the cosmetic or pharmaceutical compositions of the present invention are applied topically. Suitable compositions for topical administration include, but are not limited to, a balm, a cream, an emulsion, a gel, a hydrophilic oil, liposomes, a lotion, a mousse, a capsule, an ointment, a suspension, a solution, a salve, an impregnated dressing and any other cosmetically or pharmaceutically acceptable carrier suitable for administering the hydrophilic plant derived composition topically.

The topical formulation may be in the form of an emulsions, non-washable (water-in-oil) cream or washable (oil-in-water) cream, a gel, a lotion or a salve and the like. The cream formulation may further comprise in addition to the active compound: (a) a hydrophobic component; (b) a hydrophilic aqueous component; and (c) at least one emulsifying agent, wherein the pH of the aqueous component is in the range of from about 2.0 to about 9.0.

According to another embodiments the cosmetic or pharmaceutical compositions are formulated in the form of a solid or soft gel, selected from the group consisting of, but not limited to, an aqueous-alcoholic gel and a clear gel. Typically, the aqueous phase comprises one or more gelling agents, for example cellulose gelling agents, or synthetic gelling agents.

According to yet further embodiments the emulsions are formulated as oil in water (o/w) type emulsions, or as water in oil (w/o) type emulsions. Emulsions are defined as heterogeneous system in which two immiscible liquids are dispersed one in the other, stabilized by emulsifiers that coat the droplet to prevent droplet coalescence. Therefore, emulsions are suitable for delivering the aqueous anti-proliferative compositions of the present invention through the skin. The droplet size in such emulsions for cosmetic and medical applications is usually at the sub-micron range.

In further embodiments the cosmetic or pharmaceutical compositions of the present invention are formulated as a solution. Such a solution comprises, in addition to the active compound, at least one solvent exemplified by, but not limited to, the group consisting of, water, buffered aqueous solution and an organic solvent including ethyl alcohol, isopropyl alcohol, propylene glycol, butylene glycol, polyethylene glycol, glycerin, glycoforol, ethyl lactate, methyl lactate, N-methylpyrrolidone, ethoxylated tocopherol, dimethylsulfoxide (DMSO), tetrahydrofuran (THF), or any combination thereof.

According to yet another embodiment, the pharmaceutical composition of the present invention is formulated for oral administration. Oral formulations may be readily prepared by combining the plant derived anti-proliferative composition with pharmaceutically acceptable diluents or carriers well known in the art. Such carriers enable the compositions of the invention to be formulated as capsules, dragees, pills, tablets, gels, liquids, slurries, suspensions, syrups and the like, for oral ingestion by a patient.

Preferable amounts of the anti-proliferative aqueous composition in the cosmetic or pharmaceutical composition, the administration regime and the mode of application will depend on parameters associated with the phenomena to be treated as well as on characteristics of the treated individual (age, size, gender, etc.).

The primary function of the anti-proliferative agent of the present invention is to induce dormancy in plant meristems. As described herein above, factors that control dormancy play an important role in the industrial production of agricultural goods. The compositions comprising at least one anti-proliferative agent according to the present invention can be used to reduce the rate of plant cell proliferation when such reduction is beneficial, for example, in reducing the rate of lawn growth and therefore reducing mowing frequency and water consumption, in weed control and in preservation of fresh produce.

According to yet another aspect the present invention provides an agricultural composition comprising as an active ingredient a composition comprising at least one plant derived, anti-proliferative agent, wherein the anti-proliferative agent is a water soluble, small organic molecule; induces or maintains dormancy in at least one organ of the plant; inhibits exogenic cell proliferation; and its inhibitory activity is reversible, further comprising a suitable diluent, carrier, or surfactant, optionally further comprising at least one additional active ingredient agent selected from the group consisting of a herbicide, a pesticide, and a nutrient. According to certain embodiments, the at least one anti-proliferative agent has a molecular weight of less than about 5,000 Dalton. According to additional embodiments, the anti-proliferative agent is heat stable.

Agricultural compositions may be formulated for foliar application or for application by irrigation by methods known to one skilled in the art.

According to yet another embodiments the present invention provides a method for caring for, making up and protecting the human skin.

According to a further aspect, the present invention provides a method for at least caring for, making up or protecting the human skin comprising applying to the skin a cosmetic composition comprising an anti-proliferative aqueous composition according to the present invention. It is to be understood that the amount of the plant derived anti-proliferative aqueous composition within the cosmetic composition depends on the intended use and on parameters related to the user (e.g. age and application regime).

According to one embodiment, application of the cosmetic composition results in reduced aging signs, reduced wrinkles, promotion of skin firmness, reduced skin sensitivity, and reduced skin irritability. In another embodiments, the skin is protected against aging and external aggressions. According to one embodiment, the external aggression is one of the group consisting of, but not limited to, radiation, sun radiation, ozone, acid rain, extreme temperature, transport pollutants, industry pollutants, cleaning material, drugs, toxins or any combinations thereof.

According to a further aspect the present invention provides a method for slowing cell proliferation comprising contacting the cells with a cosmetic composition comprising an anti-proliferative aqueous composition according to the present invention in an amount effective in reducing cell proliferation.

According to one embodiment, slowing cell proliferation is beneficial for at least one phenomenon selected from the group consisting of, but not limited to, reducing undesired hair growth, reducing nail growth, obtaining better scar formation, reducing alopecia, reducing skin sebum, enhancing skin whitening and extending the duration of a tan.

According to yet a further aspect the present invention provides a method for the treatment of undesired or deleterious cell proliferation, the method comprising the step of administering to a subject in need thereof a therapeutically effective amount of a pharmaceutical composition containing an anti-proliferative aqueous composition according to the present invention.

According to one embodiment, the undesired or deleterious cell proliferation is associated with a disease or disorder selected from the group consisting of, but not limited to, malignant cell proliferation, psoriasis, seborrheic keratosis, fibrosis, restenosis and wart and/or papilloma infection.

According to one embodiment, the pharmaceutical composition of the present invention is administered in combination with at least one known anti-tumor treatment.

According to one embodiment, the additional anti tumor treatment is selected from the group consisting of radiation therapy, chemotherapy, immunotherapy, hormonal therapy and genetic therapy.

According to one preferred embodiment the pharmaceutical composition of the present invention is administered for the treatment of carcinoma or melanoma, alone or in combination with at least one additional anti-cancer agent.

According to another embodiment, the pharmaceutical composition according to the present invention is administered to inhibit proliferation of hyperproliferative mammalian cells with drug-resistant phenotypes, including multi-drug resistant phenotypes.

The present invention is explained in greater detail in the description, figures and claims below.

### BRIEF DESCRIPTION OF THE DRAWINGS

FIG. 1 shows inhibition of plant tissue proliferation by anti-proliferative composition obtained from dormant narcissus bulbs.

FIG. 2 shows inhibition of plant tissue proliferation by anti-proliferative composition obtained from Leucojum aestivum.

FIG. 3 shows inhibition of plant tissue proliferation by anti-proliferative composition obtained from orange or sweet grapefruit.

FIG. 4 shows inhibition of plant tissue proliferation by anti-proliferative composition obtained from tomato fruit.

FIG. 5 shows inhibition of plant tissue proliferation by anti-proliferative composition obtained from pitaya fruit.

FIG. 6 shows inhibition of plant tissue proliferation by anti-proliferative composition obtained from corn or wheat dormant seeds.

### DETAILED DESCRIPTION OF THE INVENTION

The present invention discloses plant-derived compositions comprising compounds capable of inducing or maintaining dormancy in a plant organ and their use as anti-proliferative compositions, for the treatment of undesired or deleterious cell proliferation.

Dormancy is a phenomenon that plays an important role in a plant life cycle, enabling the plant to survive under unfavorable environmental conditions. Entering into the phase of dormancy is involved in slowing or completely arresting meristemic cell proliferation and organ growth. Surprisingly, as discloses in the present invention, compounds that induce dormancy in plants inhibit the proliferation of exogenic cells, including plant and mammalian cells, specifically human cells.

As used herein, dormancy is a physiological state wherein growth of a meristemic tissue is slowed or ceased even though the environmental conditions may be favorable for growth.

“Induction of dormancy” or “dormancy induction” refers to providing the necessary environmental and/or physiological conditions required by a tissue to enter into a dormant state, which results in altering the growth rate of meristemic cells such that cell proliferation is slowed or ceased. The term “maintaining of dormancy” or “dormancy maintenance” refers to providing the necessary environmental and/or physiological conditions required to maintain the dormant rate of cell proliferation.

As used herein, meristemic tissue is a plant-undifferentiated tissue from which new cells are formed, e.g. the tip of a root or a stem.

As used herein, the terms “plant organ” and ”plant part” are used herein interchangeably, and refer to a structural part of a plant, for example a leaf, a root, a seed, a bud etc.

As used herein, anti-proliferative agents according to the present invention are plant derived compounds which are capable to induce and/or maintain dormancy in a plant organ, and which are capable to slow or inhibit proliferation of a plant cell as well as of a mammalian cell, including a human cell.

As used herein, exogenic cells are cells that are of different origin as the cells from which the anti-proliferative agents of the present invention are obtained.

As defined herein the term “water-soluble” agent refers to a compound that typically has solubility in water in the range of 1 gr/ml to 1 gr/30 ml at room temperature. The term “poorly water-soluble” agent as used herein refers to a compound that typically has solubility in water in the range of 1 gr/30 ml to 1 gr/10,000 ml at room temperature. The term “water-insoluble” agent refers to a compound that typically has solubility in water of less than 1 gr/10,000 ml at room temperature.

As used herein, the term “heat stable” with regard to the anti-proliferative agents of the present invention refers to an agent retaining at least 90%, preferably at least 95%, more preferably 100% of its anti-proliferative activity after heating to a temperature of from about 70° C. to about 100° C. for about 20 min.

According to certain embodiments, the present invention provides anti-proliferative compositions comprising at least one plant-derived, water-soluble anti-proliferative agent.

According to one aspect the present invention provides an anti-proliferative composition comprising at least one plant derived, anti-proliferative agent, wherein the anti-proliferative agent:

(a) is a water soluble, small organic molecule;

(b) induces or maintains dormancy in at least one organ of the plant;

(c) inhibits exogenic cell proliferation; and

(d) its inhibitory activity is reversible.

According to certain embodiments, the anti-proliferative composition comprises anti-proliferative agents having an average molecular weight of less than 5,000 Dalton. According to additional embodiments, the anti-proliferative agent is heat stable.

The inhibition of exogenic cell proliferation is measured by exposing a tissue or cell culture to different concentrations of the anti-proliferative composition and measuring the proliferation rate of the normal tissue or cell culture, wherein a decrease in the proliferation rate as compared to the proliferation rate of the tissue or cell culture incubated without the anti-proliferative composition is observed.

According to one embodiment, the reduction in the proliferation rate of the exogenic cells is at least about 20%, preferably at least about 40%, more preferably at least about 60%, most preferably at least about 80% or more reduction.

The rate of exogenic cell proliferation can be measured by various methods as are known to one skilled in the art. As exemplified herein below, the anti-proliferative activity of a composition according to the present invention is first examined using plant cell cultures. Optionally, the activity is further measured using human cell cultures.

A variety of methods that measure the viability and/or proliferation of cells in vitro have been developed. Permeability assays involve staining damaged (leaky) cells with a dye and counting viable cells that exclude the dye. Counts can be performed manually using a hemocytometer and, for example, trypan blue. Counts can be also performed mechanically using a flow cytometer and propidium iodide. Alternatively, membrane integrity can be assayed by quantifying the release of substances from cells when membrane integrity is lost, e.g. lactate dehydrogenase (LDH) or ⁵¹Cr. Another commonly used methods are based on measuring the metabolic activity by cellular reduction of tetrazolium salts, which produce highly colored end products named formazan that are measured spectrophotometrically. Various tetrazolium salts may be used in these assays. One frequently used salt is MTT, (3-(4,5-dimethylthiazolyl-2)-2,5-diphenyltetrazolium bromide) a pale yellow substrate that is cleaved by living cells to yield a dark blue water-insoluble formazan salt. After solubilizing the salt, the formazan formed can easily and rapidly be quantitated in a conventional ELISA plate reader at 530-570 nm. This process requires active mitochondria, and therefore reliable in detecting only living cells. Other tetrazolium salts used are WST-8, (2-(2-methoxy-4-nitrophenyl)-3-(4-nitrophenyl)-5-(2,4-disulfophenyl)-2H-tetrazolium.monosodium salt), which produces a water-soluble formazan dye upon dehydrogenases reduction in the presence of an electron carrier, forming yellow colored formazan; WST-1, (4-[3-(4-Iodophenyl)-2-(4-nitrophenyl)-2H-5-tetrazolio]-1,3-benzene disulfonate) also reduced by dehydrogenases of viable cells to produce water-soluble formazan, read at 440 nm; and XTT (sodium 3,3′-(1-[(phenylamino)carbonyl]-3,4-tetrazolium)bis(4-methoxy-6-nitro)benzene sulfonic acid), which is reduced to orange-red formazan with a maximum absorbance at 475 nm, that can be read at wavelengths between 450 and 500 nm without a significant loss of signal.

Direct proliferation assays use DNA synthesis as an indicator of cell growth. In these assays the incorporation of radioactive or non-radioactive nucleotide analogs is measured. Commonly used analogs are 5-bromo-2′deoxy-uridine (BrdU) and [¹⁴C]thymidine. The incorporated BrdU is detected by a quantitative cellular immunoassay using monoclonal antibodies directed against BrdU.

The present invention further discloses that the inhibitory activity of the anti-proliferative composition is reversible. When the inhibited tissue is washed and placed in a suitable medium, growth is completely resumed.

Surprisingly, the present invention discloses that the anti-proliferative compositions according to the present invention are effective in inhibiting cell proliferation in human cell cultures, as exemplified herein below for normal human fibroblasts.

The anti-proliferative compositions according to the present invention may be obtained from any plant organ that produces compounds which are responsible for the entrance of a specific plant organ into the state of dormancy, or which maintain such state of dormancy. Plant organs that may be found under the sate of dormancy are seeds, apical and lateral vegetative buds, floral buds, bulbs, corms and tubers. As described herein above, dormancy may be induced or maintained by chemical compounds that are present in the dormant organ or in a tissue surrounding the dormant organ.

According to one embodiment, the anti-proliferative composition according to the present invention is obtained from a plant organ selected from the group consisting of, but not limited to, a seed, an apical and lateral vegetative bud, a floral bud, a bulb, a corm and a tuber.

According to another embodiment, the anti-proliferative composition according to the present invention is obtained from a tissue surrounding a dormant organ. Preferably, the dormant organ is a seed, and the tissue surrounding the seed is a fruit or part of a fruit.

The phenomenon of dormancy is wide spread over the plant kingdom, and it is not associated with any specific family, species, or organ of a certain plant species. Nevertheless, dormancy is most often found in tissue or tissues within the dispersal organ of a plant. As used herein the term “dispersal organ” refers to the organ by which the plant disperses its offspring. The dispersal organ can be composed only of a primary dispersal unit such as a seed or a bulb, or it can be composed of a more complex structure such as a fruit containing seeds.

Aqueous compositions obtained from candidate sources were first tested for their ability to reduce proliferation of plant tissues, either of the same plant from which they were derived or of plants of another species. Preferably, compositions shown to be active were further examined as to their ability to inhibit the proliferation of normal human cells, specifically fibroblast, as described herein below.

According to certain embodiments, the anti-proliferative composition according to the present invention is obtained from a plant dispersal organ. According to one embodiment, the dispersal organ is a bulb. According to another embodiment, the dispersal organ is a fleshy fruit. According to one currently preferred embodiment, the anti-proliferative composition is obtained from a fleshy fruit selected from the group consisting of, but not limited to, kiwi, grapefruit, pitaya and tomato.

The anti-proliferative composition can be concentrated or diluted; a more diluted composition will result in a mild anti-proliferative activity, and a concentrated composition will give a strong cytostatic activity. Inherently, the anti-proliferative composition of the present invention is non-toxic. Toxicity can be examined by any method known in the art, for example by the application of the composition to the surface of an agarose gel in contact with cells, and measuring the effect of the composition on cell lysis.

In one embodiment the anti-proliferative compositions of the present invention are formulated in a form selected from the group consisting of, but not limited to, a solution, a suspension, an emulsion and a dry soluble lyophilized powder ready for reconstitution by combination with a vehicle prior to use.

According to one embodiment the solutions and vehicles are aqueous solutions, wherein the aqueous vehicle is water, optionally further comprising at least one buffer agent, at least one preservative or a combination thereof. According to one currently preferred embodiment the pH of the aqueous solution is in the range of from about 2.0 to about 9.0.

According to another embodiment the formulation comprises lyophilized powder ready for reconstitution by aqueous vehicle. Such lyophilized powder comprises hydrophilic plant derivative and at least one cosmetically or pharmaceutically acceptable powder base such as lactose or starch.

Optionally, at least one additional ingredient selected from the group consisting of, but not limited to, a preservative and an antioxidant, can be used.

According to one embodiment the preservative is selected from the group consisting of, but not limited to, benzyl alcohol, methyl paraben, propyl paraben, sodium salts of methyl paraben, phenoxyethanol, potassium sorbate, sodium methabisulfite, chlorophenesin and compositions thereof.

According to yet another embodiments, the present invention provides cosmetic, pharmaceutical and agricultural compositions comprising as an active ingredient an anti-proliferative composition according to the present invention.

Cosmetic products that stimulate the proliferation of skin cells, in general fibroblasts or keratinocytes, have been proposed for many years as a solution to problems of skin aging. The reasoning in support of these products is based on the finding that young skin cells divide more frequently than mature skin cells, and on the observation that high cell proliferation rate results in a better looking skin. High proliferation is associated with natural peeling, wherein the outer skin is removed and the inner layer, believed to be younger skin, appears in its place.

The massive use of compounds intended to stimulate cell proliferation, particularly of hydroxyacids, the most recent fashionable substance used for stimulation of cell proliferation, generated concerns about potential risks. One potential risk is the stimulation of pathological events related to high proliferating cells, particularly to the development of cancer cells and tumors. Cancer may also develop as a consequence of the exposure of the highly proliferating cells to UV. Another concern relates to the finite capacity of cells to divide, as postulated in the Hayflick theory (Hayflick L. et al., 1961. The serial cultivation of human diploid cell strains. Exp. Cell Res 25:585-621; Hayflick L. 1975. Current theories of biological aging. Fed. Proc. 34:9-13). The Hayflick theory has recently gained support from research showing that telomere shortening along cell divisions is involved in controlling the cell life span (Bondar, A. G. et al. 1998. Extension of life span by introduction of telomerase into normal human cells. Science 279:349-352).

The reduced capacity for cellular division in older donors and in patients subject to premature aging (e.g. in Werner syndrome and progeria) reinforces the idea that a tissue may undergo a limited number of cell divisions.

The compositions and methods of the present invention are aimed at inhibiting cellular divisions, employing the concept that inhibition of cellular divisions, rather then stimulation, should give a better answer for skin protection against aging and external aggressions.

According to one embodiment the present invention provides a cosmetic composition comprising as an active ingredient an anti-proliferative composition according to the present invention, further comprising a cosmetically acceptable diluent or carrier, optionally further comprising at least one agent selected from the group consisting of, but not limited to, a preservative, a thickener, a dispersing agent, an emulsifier, a colorant a perfume or any combination thereof, optionally further comprising at least one active ingredient selected from the group consisting of, but not limited to, an anti-oxidant, an anti-inflammation agent, a moisturizer, a vitamin, a carotenoid, a UV absorbing agent a UV protecting agent or any combination thereof.

Cosmetic application of the compositions of the present invention, intended for care of facial and body skin, advantageously uses the reversible mode of action of the anti-proliferative agents. In the long term, inhibiting cell proliferation prolongs the life span of the skin as described above, and, in the short term, provides means for complete maturation of the cells. Other cosmetic applications such as reducing the rate of hair or nail growth, prolonging the duration of a tan and enhancing skin whitening, may also take advantage of the non-toxic nature of the inhibitory activity of the anti-proliferative compositions according to the present invention. Reduced rate of epidermal cell proliferation also contributes to the firmness of the skin, as it prevents the formation of excess skin by controlling the lateral epidermal expansion.

For dermatological and pharmaceutical use, compositions comprising the plant derived anti-proliferative compositions of the present invention at higher concentrations are generally required. It is a common practice that a medicament should be applied in a regime where few applications per day for a certain period is required; however, a permanent relief of the symptoms is expected after completing the treatment regime. Therefore, the treatment of non-desired or deleterious cell proliferation, for example for the treatment of psoriasis, seborrehic keratosis, fibrosis, restenosis, wart infection, malignant cell proliferation and the like, requires the use of higher concentrations of the anti-proliferative composition. It should be noted that the above-described division of compositions for cosmetic or pharmaceutical use is somewhat artificial inasmuch as the activity may be determined by the amount of the composition or its concentration. In certain situations, the concentration and duration of use might be guided by the results obtained during treating.

The anti-proliferative characteristic of the compositions according to the present invention and their reversible mode of action are of significant value in therapeutic use for the treatment of undesired and deleterious hyper-cell proliferation.

According to one embodiment, the present invention provides a pharmaceutical composition comprising as an active ingredient a therapeutically effective amount of an anti-proliferative aqueous composition according to the present invention, further comprising a diluent, excipient or carrier.

Preferable amounts of the anti-proliferative aqueous composition of the present invention in the pharmaceutical composition, the administration regimes and the mode of application will depend on parameters associated with the phenomena to be treated as well as on characteristics of the treated individual (age, size, gender, etc.). Nevertheless, the concentration of the anti-proliferative composition is determined according to the effect requested.

Representative Formulation Forms

The cosmetic and pharmaceutical compositions of the present invention are typically formulated in a topical form selected from the group consisting of, but not limited to, balm, cream, emulsion, gel, hydrophilic oil, liposomes, lotion, mousse, capsule, ointment, suspension, solution, salve, and any other cosmetically or pharmaceutically acceptable carrier suitable for administration of the hydrophilic plant derivatives topically.

In certain embodiments the topical formulation is selected from the group consisting of, but not limited to, emulsions, non-washable (water-in-oil) creams or washable (oil-in-water) creams, a gel, a lotion or a salve and the like.

As is well known in the art the physico-chemical characteristics of the carrier may be manipulated by addition a variety of excipients, including but not limited to thickeners, gelling agents, wetting agents, flocculating agents, suspending agents and the like. These optional excipients will determine the physical characteristics of the resultant formulations such that the application may be more pleasant or convenient. It will be recognized by the skilled artisan that the excipients selected, should preferably enhance, and in any case must not interfere with the storage stability of the formulations.

According to certain embodiments the emulsion formulation comprising in addition to the active compound: (a) a hydrophobic component; (b) a hydrophilic aqueous component; and (c) at least one emulsifying agent.

As a non-limiting example the hydrophobic component of the emulsion is present in an amount from about 10% to about 90% (w/w) based on the total weight of the composition, preferably in an amount from about 20% to about 80% (w/w) based on the total weight of the composition.

The hydrophobic component of the emulsion is exemplified by the group consisting of, but not limited to, mineral oil, yellow soft paraffin, white soft paraffin, paraffin, hydrous wool fat, wool fat, wool alcohol (lanolin alcohol), petrolatum and lanolin alcohols, beeswax, cetyl alcohol, almond oil, arachis oil, castor oil, cottonseed oil, ethyl oleate, olive oil, sesame oil, and mixtures thereof.

The hydrophilic aqueous component of the emulsion is exemplified by water alone or alternatively any cosmetically or pharmaceutically acceptable buffer or solution.

Exemplary buffers are borate (borax), citrate, acetate, phosphate and mixtures thereof. The hydrophilic aqueous component of the emulsion may be present in an amount from about 10% to about 90% (w/w) based on the total weight of the composition, preferably in an amount from about 20% to about 80% (w/w) based on the total weight of the composition.

Emulsifying agents may be added in order to stabilize the emulsion and to prevent the coalescence of the drops. The emulsifying agent reduces the surface tension and forms a stable, coherent interfacial film. For example, the emulsifying agent is a complex emulsifier which comprises a combination of a hydrophilic and a hydrophobic emulsifying agent. The complex emulsifier is typically present in an amount effective to stabilize the emulsion formed from the hydrophobic component and hydrophilic aqueous component. The ratio of the hydrophilic and hydrophobic emulsifying agents comprising the complex emulsifier depends on the type of emulsion formulated (i.e. oil-in-water and water-in-oil) and on the required HLB (hydrophilic-lipophilic balance) of the inner emulsified phase. As an example, the concentration of the complex emulsifier is in the range from about 2% to about 40% (w/w) based on the total weight of the composition. The complex emulsifier is exemplified by, but not limited to emulsifying wax, cetrimide emulsifying wax, cetomacrogol-emulsifying wax and Lanette wax SX. The complex emulsifier may be formed in-situ by the reaction of triethanolamine or an alkaline substance and oleic acid, or by the reaction of triethanolamine or an alkaline substance and stearic acid.

Suitable hydrophilic emulsifying agents comprising the complex emulsifier may be selected from the group consisting of, but not limited to, polyoxyethylene sorbitan monolaurate (Tween 20), polyoxyethylene sorbitan monopalmitate (Tween 40), polyoxyethylene sorbitan monostearate (Tween 60), polyoxyethylene sorbitan monooleate (Tween 80), plyoxyethylene lauryl ether (Brij 35), polyoxyethylene castor oil (Atlas G-1794), sodium lauryl sulfate, cetrimide, cetomacrogol and mixtures thereof.

Suitable hydrophobic emulsifying agents comprising the complex emulsifier may be exemplified but not limited to the group consisting of, but not limited to, sorbitan trioleate (Span 85, Aracel 85), sorbitan tristearate, (Span 65), sorbitan monooleate (Span 80), propylene glycol monostearate, sorbitan sequioleate (Aracel C), glycerol monostearate, propylene glycol monolaurate (Atlas G-917, Atlas G-3851), sorbitan monostearate (Span 60, Aracel 60), sorbitan monopalmitate (Span 40, Aracel 40), sorbitan monolaurate (Span 20, Aracel 20), cetostearyl alcohol, cetyl alcohol, oleic acid, stearic acid and mixtures thereof.

A suitable emulsifying agent may be exemplified by, but not limited to, the group consisting of cholesterol, cetostearyl alcohol, wool fat (lanolin), wool alcohol (lanolin alcohol), hydrous wool fat (hydrous lanolin), and mixtures thereof.

As an example, the concentration of the at least one emulsifying agent is in the range from about 2% to about 40% (w/w) based on the total weight of the composition.

According to another embodiments the compositions of the present invention are formulated in a form of a gel further comprising at least one gelling agent. Suitable gelling agents may be exemplified by, but not limited to, the group consisting of hydrophilic polymers, natural and synthetic gums, crosslinked proteins and mixture thereof. Typically, the polymers are selected from the group consisting of, but not limited to, hydroxyethylcellulose, hydroxyethyl methylcellulose, methyl cellulose, hydroxypropylcellulose, hydroxypropyl methylcellulose, carboxymethyl cellulose, and similar derivatives of amylose, dextran, chitosan, pullulan, and other polysaccharides; crosslinked proteins such as albumin, gelatin and collagen; acrylic based polymer gels such as Carbopol, and hydroxyethyl methacrylate based gel polymers, polyurethane based gels and mixtures thereof.

The gums may be selected from the group consisting of, but not limited to, acacia, agar, carageenan, dextrin, gelatin, guar gum, hyaluronic acid, tragacanth gum, xanthan gum, and mixtures thereof. As an example, the gelling agent is present in an amount from about 1% to about 25% (w/w) based on the total weight of the composition. The pH of the aqueous phase of the gel is typically in the range of from about 2.0 to about 9.0.

In yet another embodiments cosmetic or pharmaceutical compositions of the present invention may be formulated as a solution. Such a solution comprises, in addition to the active compound, at least one solvent exemplified but not limited to the group consisting of, but not limited to, water, buffered solutions, organic solvents such as ethyl alcohol, isopropyl alcohol, propylene glycol, butylene glycol, polyethylene glycol, glycerin, ethyl lactate, methyl lactate, N-methylpyrrolidone, ethoxylated tocopherol, dimethylsulfoxide (DMSO), tetrahydrofuran (THF), or any combination thereof.

According to one embodiment the solution comprises a mixture of the active compound in an aqueous solution of a pH range between about 2.0 and about 9.0. The solutions may be maintained as a mixture of hydrophilic components or contain water at various amounts for topical use.

The topical composition of the present invention may optionally contain at least one additional ingredient, selected from the group consisting of, but not limited to, a preservative, an antioxidant, humectants, an emollient, a thickener, a structuring agent, a stabilizer, a coloring agent, and a perfume.

According to yet another embodiment, the pharmaceutical composition of the present invention is formulated for oral administration. Oral formulations may be readily prepared by combining the anti-proliferative composition with pharmaceutically acceptable diluents or carriers well known in the art. Such carriers enable the compositions of the invention to be formulated as capsules, dragees, pills, tablets, gels, liquids, slurries, suspensions, syrups and the like, for oral ingestion by a patient.

Solid forms for oral administration include capsules, tablets, pills, powders and granules. In such solid forms, the active compound is admixed with at least one inert diluent, such as sucrose, lactose or starch. Such oral forms can also comprise additional substances other than inert diluent. In the case of capsules, tablets and pills, the formulation may also comprise buffering agents. Tablets and pills can additionally be prepared with an enteric coating.

Liquid forms for oral administration include pharmaceutically acceptable emulsions, solutions, suspensions, syrups and elixirs, containing inert diluents commonly used in the pharmaceutical art. Besides inert diluents, such compositions can also include adjuvants, such as wetting agents, emulsifying and suspending agents, and sweeteners.

Preferred Uses of the Anti-Proliferative Compositions

According to yet another aspect the present invention provides a method for at least caring for, making up and protecting the human skin, the method comprising the step of applying to the skin a cosmetic composition containing as an active ingredient an anti-proliferative aqueous composition according to the present invention.

Skin is subjected daily to numerous negative environmental factors and pollutants. These pollutants include, but are not limited to, atmospheric factors, chemical pollutants and biological pollutants. Examples of atmospheric factors that affect the skin include, but are not limited to, radiation such as UV radiation from the sun, ozone, acid rain and extreme temperatures. Chemical and biological pollutants include pollutants from cars, industry, free radicals, cleaning materials, drugs and toxins.

As described herein above, cells often have a limited capacity to replicate. Therefore, slowing cell proliferation prolongs their life span. Moreover, slowing the proliferation process provides means for complete maturation of the cells. Thus, slowing the proliferation of epidermal skin cells not only has an antiaging effect, as it preserves the cell ability to divide for longer time periods, but it also results in healthier cells. Mature, properly differentiated epidermal cells have a better ability to protect inner cell layers from environmental aggression.

According to one embodiment, the external aggression is selected from the group consisting of, but not limited to, radiation, sun radiation, ozone, acid rain, extreme temperature, transport pollutants, industry pollutants, cleaning material, drugs, toxins or any combinations thereof.

According to a further aspect the present invention provides a method for slowing cell proliferation, the method comprising the step of contacting the cells with a cosmetic composition containing anti-proliferative aqueous composition in an amount effective in reducing cell proliferation.

According to one embodiment, slowing cell proliferation is beneficial for at least one phenomenon selected from the group consisting of, but not limited to, reducing undesired hair growth, reducing nail growth, obtaining better scar formation, reducing alopecia, reducing skin sebum, enhancing skin whitening and extending the duration of a tan.

Scalp baldness (alopecia) is one of the phenomena associated with aging of the skin in an individual. In individuals suffering from alopecia, the life span of scalp hair decreases substantially (e.g. from a life span of about 3 years in a normal individual to a life span of about one year in an individual suffering from alopecia). Therefore, decreasing the rate of hair growth in an individual having a high probability of developing alopecia, or in an individual already showing for signs of scalp hair loss, will decrease the extent of such hair loss. Administration of the cosmetic compositions of the invention, which comprise anti-proliferative agents to such an individual, will be beneficial for reduction or prevention of hair loss.

An additional phenomenon that may be treated by administration of the cosmetic compositions according to the present invention is associated with overgrowth of hair in various parts of an individual's body (Hirsutism), including arms, back, etc. Such undesired overgrowth of hair appears many times in aging individuals and, at times, is associated with loss of scalp hair in the same individual. Due to their ability to reduce cell growth, compositions of the invention may be useful in reducing such undesired overgrowth of hair.

In addition, the cosmetic compositions according to the present invention may be useful as a complementary agent administered in combination with or following hair removal treatments such as, for example, shaving (where said extract may be incorporated in an aftershave solution) or hair stripping.

The cosmetic compositions of the present invention may also be useful for extending the duration of a tan in an individual. Following exposure to the sun, epidermal cells comprise a high concentration of melanin. During skin renewal such melanin comprising cells are shed. By slowing the cell renewal process in the skin, the melanin comprising cells and thus the tan remain for a longer period of time.

Surprisingly, the anti-proliferative compositions of the present invention were also found to be useful for enhancing skin whitening. Pigmentation and hyper-pigmentation of the skin is due to melanin accumulation. Melanin accumulation is due to two processes: melanin production via the melanin synthesis pathway, in which the activity of tyrosinase is the limiting factor; and proliferation of the melanin containing cells—the melanocytes. The quantity of melanin in cultured melanoma cells was reduced in the presence of narcissus-derived anti-proliferative composition of the present invention. It was found that the reduction in the melanin content resulted from the reduction in melanocyte cell number, while the melanin synthesis per cell was not affected. Thus, the anti-proliferative compositions of the present invention can regulate the overall content of melanin in a certain tissue by regulating the number of melanin-producing cells.

The amount of the cosmetic composition comprising at least one anti-proliferative agent to be administered for the above indications, the administration regimes as well as their mode of application will depend both on characteristics of the treated individual (age, size, gender, etc.) as well as on parameters associated with the phenomena to be treated (such as the extent of scalp hair loss, the specific body parts in which there is overgrowth of hair, etc.).

According to one currently preferred embodiment, the cosmetic compositions of the present invention to be used for the treatment of the above-described indications are applied topically.

According to yet a further aspect the present invention provides a method for the treatment of undesired or deleterious cell proliferation, the method comprising the step of administering to a subject in need thereof a therapeutically effective amount of a pharmaceutical composition containing an anti-proliferative aqueous composition according to the present invention.

According to one embodiment, the undesired or deleterious cell proliferation is associated with a disease or disorder selected from the group consisting of, but not limited to, malignant cell proliferation, psoriasis, seborrehic keratosis, fibrosis, restenosis and wart and/or papilloma infection.

Due to their significant anti-proliferative effect, the therapeutic compositions according to the present invention are beneficial for the treatment of various malignancies. The rate of cell division is a significant factor in determining the probability of a cell to become a premalignant or malignant cell. In addition, as known, the formation of a benign or malignant tumor is dependent, inter alia, on continuous divisions of the cells forming the tumor. Administration of the anti-proliferative therapeutic compositions of the present invention to an individual at early stages of the formation of a benign or malignant tumor will delay the tumor growth, resulting in reduction of the tumor load and in alleviation of the tumor-related symptoms. Said therapeutic compositions may be effective in the treatment of primary as well as secondary (metastatic) tumors.

According to one embodiment, the pharmaceutical composition of the present invention is administered in combination with at least one known anti-tumor treatment.

According to one embodiment, the additional anti tumor treatment is selected from the group consisting of, but not limited to, radiation therapy, chemotherapy, immunotherapy, hormonal therapy and genetic therapy.

According to one preferred embodiment, the additional anti tumor treatment is chemotherapy.

Some of the most effective and commonly used chemotherapy agents, including but not limited to taxol, gemacetabin, vinca alkaloids and many others, are known to affect cancer cells in a specific stage of the cell cycle. These agents may therefore be described as “cell cycle specific agents”. The cell cycle can be described as a sequence of phases through which the cell proceeds as it proliferates. The phases of this cycle are denoted G1, S, G2 and M, where G1 is the gap preceding synthesis of DNA, S is the phase during which the cell synthesizes DNA, G2 is the gap between the S phase and division or mitosis (M). Cells that are not proliferating may be arrested in a stage referred to as G₀.

Without wishing to be bound to a specific mechanism, exposure of malignant cells to the pharmaceutical composition comprising anti-proliferative agents according to the present invention arrests the cell cycle, whereas its removal enable the cancer cells to regain their normal cycling. Effectively, this serves to synchronize the cells, thus bringing a larger proportion of the malignant cells to the specific stage of the cell cycle where they are sensitive to the effects of the chemotherapeutic agent. As a result, toxic side effects due to the influence of the chemotherapeutic treatments on normal cells may be significantly reduced and when beneficial, higher concentrations of the chemotherapeutic treatments may be used.

According to one preferred embodiment the pharmaceutical composition of the present invention is administered for the treatment of carcinoma or melanoma, alone or in combination with at least one another anti-cancer agent.

According to another embodiment, the pharmaceutical composition according to the present invention is administered to inhibit proliferation of hyperproliferative mammalian cells with drug-resistant phenotypes, including multi-drug resistant phenotypes.

According to yet another embodiment, application of the therapeutic compositions according to the present invention is beneficial for the inhibition of fibrosis, e.g. skin fibrosis, cirrhosis, and others, associated with fibroblast proliferation. The anti-proliferative agents of the present invention, effective in reducing fibroblast proliferation, provide effective, non-toxic treatment for fibrosis. In a similar manner, the therapeutic compositions of the present invention may also be useful in the treatment of psoriasis, which results from over proliferation of keratinocytes. Seborrheic keratosis, papilomas and warts may also be treated by the therapeutic compositions.

Another application of the therapeutic composition may involve its administration to an individual during the period in which a scar is formed, e.g. after an operation, in order to decrease scar formation. By slowing the rate of cell proliferation during the healing process, the final scar may be less apparent. In addition, the anti-fibrotic effect of the therapeutic compositions according to the present invention decreases the formation of cheloids, which frequently appear after healing.

The anti-proliferative aqueous compositions according to the present invention contain at least one anti-proliferative agent that its primary activity is to arrest proliferation of plant cells. The present invention discloses that such composition are active when applied to plant cells from the same plant origin of which they were derived, as well as when applied to cells of plants from another origin. Therefore, the anti-proliferative compositions of the present invention can be used to reduce the rate of plant cell proliferation when such reduction is beneficial, for example, in reducing the rate of lawn growth and therefore reducing mowing frequency, in weed control and in preservation of fresh produce.

According to a further aspect, the present invention provides an agricultural composition comprising as an active ingredient an anti-proliferative aqueous composition in an amount suitable to arrest the growth of exogenic plant cell or tissue, further comprising a suitable diluent, carrier, or surfactant, optionally further comprising at least one additional active ingredient selected from the group consisting of a pesticide, a fungicide, an anti-biotic agent, a herbicide a nutrient or any combination thereof. Agricultural compositions may be formulated for foliar application or for application by irrigation by methods known to one skilled in the art.

The principle of the invention, employing compounds that are capable to induce or maintain dormancy in a plant part as anti-proliferative agents may be better understood with reference to the following non limiting examples.

### EXAMPLES

### Example 1

### Production of Anti-Proliferative Aqueous Compositions

Production of an Anti-Proliferative Composition by Aqueous Extraction

The protocol for extracting the anti-proliferative agents of the present invention from a dry dormant plant material include several general steps, which can be modified according to the specific plant material used as described herein below:

(1) Harvesting of dormant plant material. Suitable conditions should be kept after harvesting as to maintain the plant material in the state of dormancy. For example, Narcissus bulbs were kept for 30 days at 28° C.

(2) The dormant dry material is washed in tap water. If necessary, the outer surface is removed. For example, Narcissus bulbs were peeled.

(3) The clean material is crushed, water is added and the mixture is homogenized. For example, Narcissus bulbs or Leucojum aestivum bulbs were mixed with water at a ratio of 3:7 (bulbs:water). The homogenized mixture is then incubated in room temperature to enable extraction. For example, the homogenized mixture of Narcissus or Leucojum aestivum was incubated for 30 min.

(4) Large debris is then separated from the aqueous extract. For Narcissus and Leucojum aestivum, separation was performed through a sieve.

(5) Starch separation for high-starch containing plant material. For example, extract of Leucojum aestivum was incubated at 4° C. for 2 hours, and then centrifuged at 4500 rpm for 20 min to remove starch.

(6) Protein removal is performed by heating the aqueous extract and centrifugation. Narcissus extract was heated to 105° C. for 1 h; the resulted solution was centrifuged and the supernatant was heated again to 105° C. for 30 min. Leucojum aestivum extract was heated to 105° C. for 2 h; the resulted liquid was centrifuged and the supernatant was heated again to 105° C. for 30 min.

(7) The resulted solution is cooled to 60° C., preservative is added, and the solution is centrifuged again. The supernatant is collected and the batch is standardized by addition of water.

(8) Optionally, the solution is ultrafiltrated. The ultrafiltration was performed using a 5,000 Dalton cutoff membrane (Osmonics Inc.).

(9) The solution is filter-sterilized as to obtain the anti-proliferative composition of the invention, designated as “Dormin”. The Narcissus and Leucojum aestivum extracts were filter-sterilized through 0.2-micron filter.

Production of an Anti-Proliferative Composition from a Fleshy Fruit

Separating the anti-proliferative agent-containing fraction from a fleshy fruit is performed by a general procedure according to the steps listed below, which are modified according to the specific fruit type used.

(1) Obtaining the aqueous fraction from the fruit. The procedure depends on the fruit type: fruit having thick pericarp, for example pitaya fruit (Hylocereus undatus), were first squeezed and then crushed and homogenized. Fruit having thin pericarp like tomato, were directly crushed and homogenized.

(2) Seeds, pulp and other debris are removed from the liquid, typically designated herein as “serum”.

(3) Further sediments are separated by centrifugation.

(4) The serum is further cleared by heating. Serum obtained from pitaya fruit was heated twice, and debris was removed after each heating by centrifugation. Tomato serum was heated once to 75° C. in the presence of 1% active carbon to decolorize and deodorize the aqueous solution. Further sediments were separated by centrifugation.

(5) The supernatant is collected and sterile-filtered. Optionally, preservatives are added. The serum is then filter sterilized to obtain the anti-proliferative composition of the invention. Serum obtained from Pitaya and tomato fruit was filtered through 0.2 micron filter.

(6) Optionally, the solution is ultrafiltrated before the sterilizing filtration, using a 5,000 Dalton cutoff membrane (Osmonics Inc.).

### Example 2

### Evaluation of the Anti-proliferative Activity of the Composition—Inhibition of Cell Proliferation in a Plant Tissue

In plants, the proliferation of a meristemic tissue, an embryo within a seed, was examined. Such an embryo can grow to a plant, comprising root as well as hypocotyl tissues. Inhibition of root elongation was thus used as a test for the anti-proliferative activity of the compositions of the present invention, according to the protocol described below.

Materials: Cucumber seeds (vr. “Kfir”, or “Delila” Zeraim Gedera, Israel, 99.9% clean, at least 90% germination); Tap-water; Filter paper; Petri-dishes (15 cm diameter); Plastic Trays; Plastic Beaker; Strainer; Ruler; Incubator.

Procedure: Seeds in an amount sufficient for covering two plastic trays were washed with running tap water for 20 minute. After the washing, water was removed from the seeds as much as possible. Filter paper to cover each tray was wetted with 60 ml of water and placed on the plastic tray. The washed seeds were spread on top of the paper in the tray. Another tray was placed on top of the tray as to cover it, and both trays were placed within a plastic bag. The trays were placed inside an incubator set on 28° C., 46-50% RH. The seeds were incubated for 18-24 hours, until a root tip of about 2 mm emerged from about 90% of the seeds.

A series of dilutions of the examined extract were prepared as follows:

| % Extract | Extract volume (ml) | Tap-water volume (ml) |
|---|---|---|
| 0 | 0.0 | 10.0 |
| 2.5 | 0.25 | 9.75 |
| 5.0 | 0.50 | 9.50 |
| 10 | 1.0 | 9.0 |
| 20 | 2.0 | 8.0 |

5 ml of each dilution were poured into 2 Petri dishes. A filter paper was placed in each Petri dish and wetted with the extract. 12 pre-germinated seeds were placed in each plate (2×12=duplicates). The plates were incubated for 48 hours at 28° C.

After 48 hours of incubation, the seeds were removed from the dishes and the root and/or hypocotyl length (mm) was measured using a ruler. The average percentage of inhibition for each extract dilution was calculated as follows:
% Inhibition=(Lo_{O}−L_{E})/L_{O}*100

L_{O}—mean lengths of roots emerged from seeds incubated with 0% extract

L_{E}—mean lengths of root emerged from seeds incubated with each extract dilution. A plot of the inhibitory activity as a function of the extract concentration was drawn.

Results

Anti-Proliferative Activity of Bulb Derived Anti-Proliferative Composition

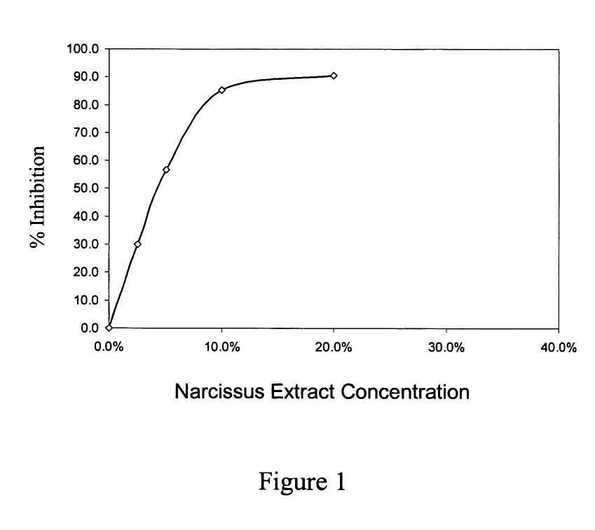

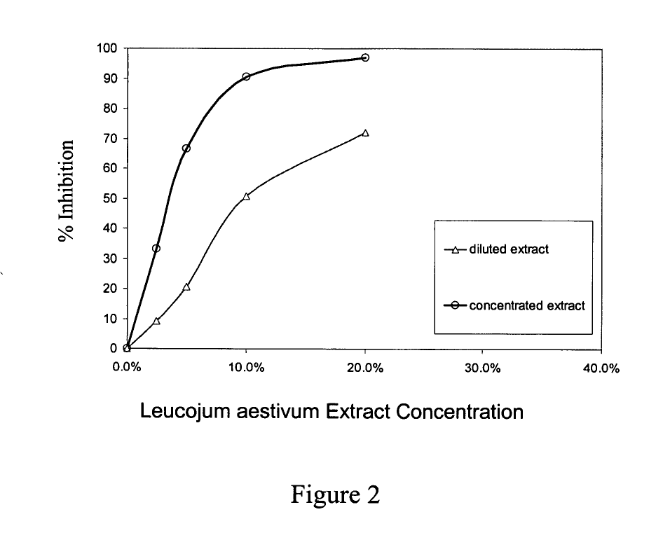

Narcissus-derived anti-proliferative composition was prepared as described in Example 1, and its activity was evaluated by examining root elongation as described in Example 2 above. FIG. 1 shows the anti-proliferative effect of an extract obtained from dormant narcissus bulbs, demonstrated by inhibition of root tip elongation as described hereinabove. Similarly, FIG. 2 shows the anti-proliferative activity of extract obtained from dormant bulbs of Leucojum aestivum. These result demonstrate that a concentrated composition have a stronger anti-proliferative activity compared to a diluted one.

Extracts of dormant bulbs of various plants were also prepared and examined for their anti-proliferative activity. Dormant field bulbs were disinfected in soap water for a period of 1 hour. The bulbs were then cut and homogenized in distilled water (30 sec×3) using a Homogenizer Ultra-Turbo-Turax. The homogenized preparation was then filtrated through a 0.45 μm sterile filter and then through a 0.22 mm filter and the filtrate was collected. The concentration of each composition was defined as original bulb weight (gr.) per final extract volume (ml). The activity of the extracts was examined as described in Example 2 above.

As seen in Table 1 below, most of the extracts showed good inhibitory effect on the elongation of emerging cucumber roots (up to about 60% inhibition in average). Several of the bulb extracts showed very good inhibitory activity of about 90% inhibition (e.g. an extract obtained from dormant bulb of Pancratium maritumum). Several other extracts showed a low inhibitory effect which may, in some cases, be due to the fact that the extract was obtained from bulbs that were not fully dormant.

The effect of extracts obtained from bulbs of Pancratium maritumum and Hyacinth carnegie were further tested for their effect on cucumber root elongation by examining various concentrations of the extracts. The results (not shown) showed correlation between the concentration of the added extract and the inhibition effect of the extract on cell proliferation and root elongation.

TABLE 1
Anti-proliferative activity of extract obtained from various
dormant bulbs.

| | | Root elongation after 48 hours |
|---|---|---|
| Extract Source | | (% Inhibition) |
| Sparaxis | 0.52 gr./ml | 49 |
| Hyacinth carnegie | 0.40 gr./ml | 94 |
| Freesia | 0.42 gr./ml | 77 |
| Crocus | 0.41 gr./ml | 30 |
| Ornithogalum arabicum | 0.82 gr./ml | 54 |
| Montbartia | 0.64/gr./ml | 63 |
| Scilla hyacinthus | 1.25 gr./ml | 68 |
| Pancratium maritumum | 0.71 gr./ml | 93 |

Anti-Proliferative Activity of Fruit Derived Anti-Proliferative Composition

As shown in Table 2 below, a composition derived from grapefruit comprises at least one anti-proliferative agent having inhibitory activity. The composition significantly inhibited the cell proliferation of the root and hypocotyl meristemic cells.

TABLE 2
Inhibition of plant cell proliferation by grapefruit
derived composition

| | Length | % |
|---|---|---|
| | (mm) | Inhibition |
| | (After 72 h) | (After 72 h) |
| Treatment | Root | Root |
| dH₂O | 110 | — |
| Grapefruit derived anti- | 3 | 97 |
| proliferative composition | | |

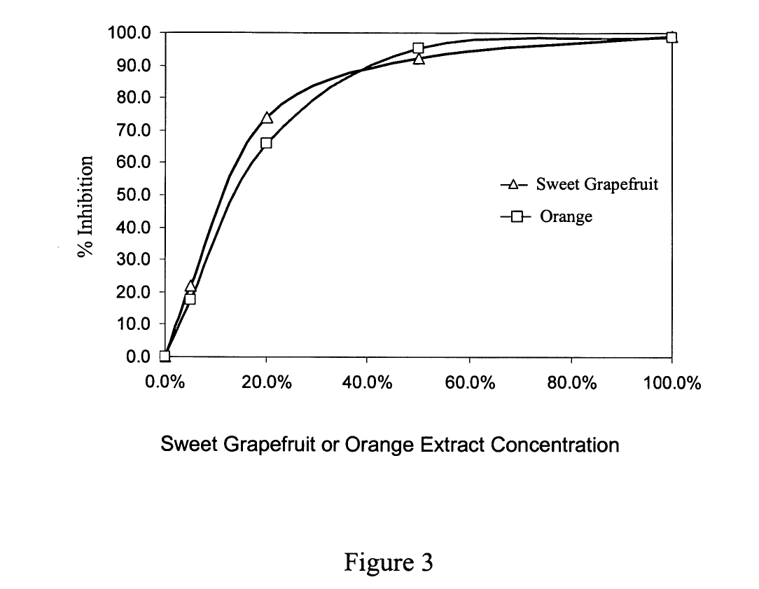

Extract from fruit of other citrus species were also examined for their anti-proliferative activity. As shown in FIG. 3, extract of sweet grapefruit as well as orange fruit were very efficient in inhibiting root elongation.

Various dilutions were prepared from the compositions obtained from grape or kiwi fruit as described above (designated KC or GC, respectively). The inhibitory activity of these dilutions on proliferation of plants cells was examined as described above. Table 3 below demonstrates that both the kiwi and the grape derived compositions significantly inhibited the growth of both cucumber roots and hypocotyls.

TABLE 3
Inhibition of plant cell proliferation by kiwi or grape derived
composition

| | Root |
|---|---|
| | % Inhibition |
| | after 48 h |
| dH₂O | 0 |
| KC 8% | 72 |
| KC 4% | 55 |
| KC 2% | 42 |
| KC 0.4% | 7 |
| GC 8.3% | 88 |
| GC 4.15% | 67 |
| GC 2.08% | 42 |
| GC 0.415% | 20 |

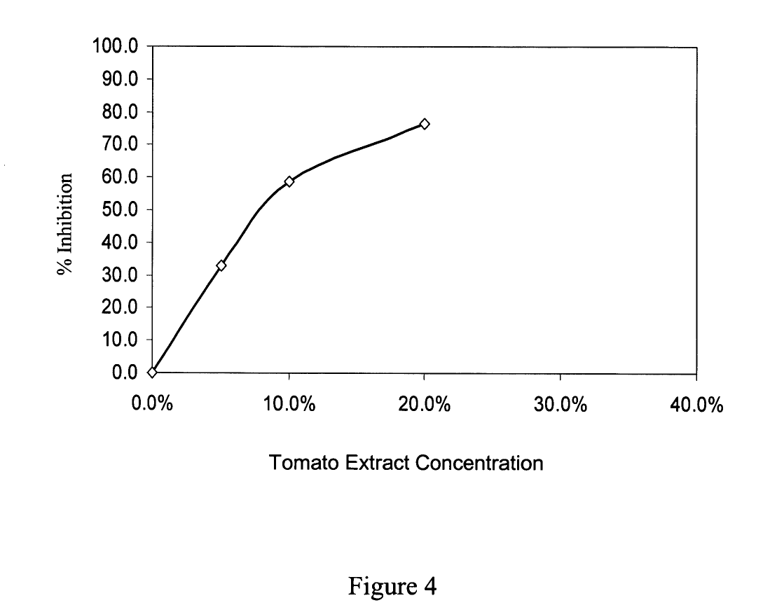

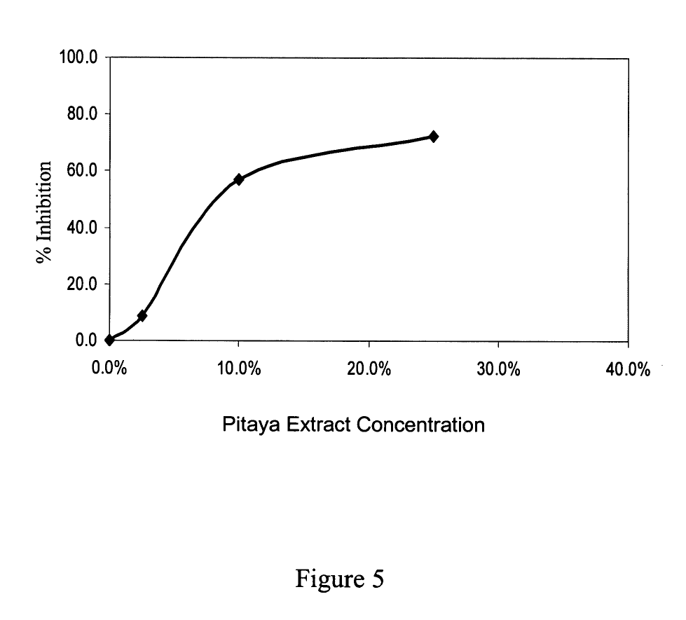

FIGS. 4 and 5 show, respectively, the inhibitory activity of tomato derived and pitaya fruit (Hylocereus undatus) derived aqueous anti-proliferative composition on plant tissue. The extracts were prepared as described in Example 1 hereinabove.

Anti-Proliferative Activity of Seed Derived Anti-Proliferative Composition

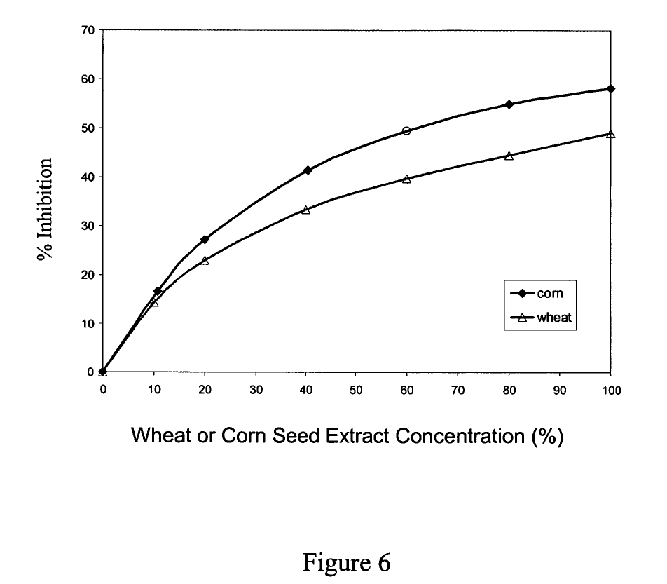

Seed extraction was performed according to the principles described in Example 1 herein above for production of anti-proliferative composition by aqueous extraction from dry dormant plant material. Wheat and corn seeds were milled to obtain a powder. The powder was mixed with water at a powder:water ratio of 1:3 for 2.5 h at room temperature. The resulted mixture was then filtered through cheesecloth, and the filtrate was incubated overnight at 4° C. After incubation, the mixture was centrifuged and the supernatant comprising the anti-proliferative agents was separated. FIG. 6 shows the anti-proliferative activity of aqueous extracts of dormant corn and wheat seeds as observed by inhibition of root growth as described herein above.

### Example 3

### Inhibition of Normal Human Dermal Fibroblasts by Anti-Proliferative Composition

Another feature of the anti-proliferative compositions according to the present invention is their capability to inhibit proliferation of mammalian cells, specifically human cells. This anti-proliferative activity of the plant derived anti-proliferative compositions of the present invention was evaluated by their effect on proliferation of normal human dermal fibroblasts (NHDF) cultured in vitro.

Test Compound: Narcissus-Derived Anti-Proliferative Composition as Stock Solution

Materials and Methods

Cells

| | |
|---|---|
| Type: | pool of normal human dermal fibroblast NHDF (pool No. |
| | R7PF2 (7^{th} passage) |
| Culture: | 37° C., 5% CO2, |
| Medium: | MEM/M199, 3:1 (Gibco 31570021/2115130); sodium |
| | bicarbonate 1.87 mg/ml (Gibco 25080060); L-glutamine |
| | 2 mM (Gibco 25030024); penicillin 50 UI/ml (Polylabo |
| | 60703); fetal calf serum 10% (v/v Gibco 10106151) |

Test Compounds
1. Narcissus derived anti-proliferative composition, lyophilized to form a powder, designated IBR-1 powder. Stock solution was prepared as 5 mg powder/ml sterile distilled H₂O. Dilution was made in sterile culture medium, as follows: 1/20 (250 μg/ml); 1/40 (125 μg/ml); 1/200 (25 μg/ml); 1/2000 (2.5 μg/ml); 1/20000 (250 ng/ml); 1/40000 (125 ng/ml); 1/200000 (25 ng/ml); and 1/400000 (12.5 ng/ml).
2. Narcissus derived anti-proliferative composition in a liquid form, designated IBR-1 liquid. The source narcissus derived anti-proliferative composition was diluted in sterile culture medium. Concentrations assayed were 1/20; 1/40; 1/200; 1/2000; 1/20000; 1/40000; 1/200000; and 1/400000.
   Assay

The assay was performed in 96 well microplates seeded with 1000 cells/well. After a 24 h pre-culture, media were changed for media containing the compound to be assayed in a selected concentration. The cells were cultured for a total period of 144 h, with one medium change (at 72 h). For each experimental condition, six replicates were performed (n=6); twelve well served as a control in each plate.

The wells were individually observed under light microscopy after incubation of 24 h, 48 h and at the end of the experiment (144 h). All these observations were collected for confirmation of viability measurements.

After 144 h of incubation, cell monolayers were rinsed and incubated for 3 h at 37° C., with fresh medium containing soluble MTT (3-(4,5-dimethylthiazolyl-2)-2,5-diphenyltetrazolium bromide). Formazan crystals produced by viable cells were then dissolved in dimethylsulfoxide and the resulting optical density was measured at 540 nm with a ThermoMax microplate reader (Molecular Devices). Data analysis was performed with the SoftMax software.

Results and Conclusions

The dose-effect profiles of the two preparations of IBR-1 were almost the same (Table 4). Both preparation types—the liquid and the lyophilized powder re-instated into a liquid solution showed cytostatic activity at the three highest concentrations tested (1/20-1/200). No effect could be detected at doses below dilution of 1/200. A slight difference towards a better activity was observed at a dilution of 1/200 for the liquid preparation.

Both the cell observation and the MTT-assay results indicate that the narcissus derived anti-proliferative composition, at the dilutions of 1/20 and 1/40, strongly reduce cell multiplication

In the culture conditions used, NHDF population normally doubles each 48 h. After 24 h treatment (dilutions 1/20 & 1/40), the cell population was slightly reduced (80% of the control), indicating that the compound was not cytotoxic (no significant cell lethality). At 48 h, the treatments with dilutions 1/20, 1/40 & 1/200 reduced the population by 50-75%. This result accords with a non-toxic blockage of cell division (the cells present at the beginning of the treatment were present, but no more division occurred; n cells in the treated wells, 2n cells in controls).

TABLE 4
Proliferation of normal human dermal fibroblasts population after
treatment with narcissus derived anti-proliferative composition

| | | | | Proliferation |
|---|---|---|---|---|
| | Cell observations | | | Index |
| | 24 h | 48 h | 144 h | (% Of control) |
| Dilution | | | | |
| (Narcissus-derived | | | | |
| anti-proliferative | | | | |
| composition, from a | | | | |
| powder source) | | | | |
| 1/20 | 80%* | 25 | 0 | 1.08 |
| 1/40 | 80%* | 50%** | 5%** | 5.84 |
| 1/200 | + | + | 50%** | 37.97 |
| 1/2000 | + | + | + | 102.5 |
| 1/20000 | + | + | + | 99.72 |
| 1/40000 | + | + | + | 96.46 |
| 1/200000 | + | + | + | 98.97 |
| 1/400000 | + | + | + | 102.00 |
| Dilution | | | | |
| (Narcissus-derived | | | | |
| anti-proliferative | | | | |
| composition, liquid | | | | |
| 1/20 | 80%* | 25 | 0 | 0.22 |
| 1/40 | 80%* | 50%** | 5%** | 3.30 |
| 1/200 | + | 50%** | 50%** | 19.51 |
| 1/2000 | + | + | + | 105.60 |
| 1/20000 | + | + | + | 100.30 |
| 1/40000 | + | + | + | 104.00 |
| 1/200000 | + | + | + | 96.77 |
| 1/400000 | + | + | + | 99.44 |

Cell Observation (columns 2-4): the apparent relative cell number (%) at different incubation times was indicated by microscopic observation.
Proliferation index (column 5) was evaluated by measuring MTT hydrolysis at the end of the experiment.
*Cells were blocked, no division seemed to occur; the cell density was apparently the same as this at the beginning of the experiment
**Cell multiplication was strongly reduced.

Test Compound: Tomato Derived Anti-Proliferative Composition as Stock Solution

Materials And Methods

Biological model

| | |
|---|---|
| Type: | Pool of normal human dermal fibroblast |
| | (NHDF 7^{th} or 8^{th} passage). |
| Culture medium: | DMEM (Life Technologies 21969035); |
| | Glutamine 2 mM (Life technologies 25030024); |
| | Penicillin 50 UI/ml; Streptomycin 50 μg/ml (Life |
| | technologies 15070063); Fetal calf serum 10% (Life |
| | Technologies 10106451). |

Dilutions

The source tomato derived anti-proliferative composition was diluted in sterile culture medium. Concentrations assayed were 1/10; 1/20; 1/40; 1/80; and 1/160 dilution of the raw tomato-derived anti-proliferative composition stock solution.

Assay

The assay was conducted using six 96-well microplates seeded with normal human fibroblasts, 1000 cells/well. The plates were maintained at 37° C., 5% CO₂ (non-confluent cultures). The protocol used is illustrated in the scheme below. After a 24 h pre-culture, the media were replaced with media containing either the dilutions of the compounds to be assayed or the medium alone as a control. The cells were cultured for a total period of 144 h, with one medium change at 72 h. Each experimental condition was performed in 6 replicates. Each well was observed by light microscopy after 48 h and at the end of the assay (144 h). These observations were gathered to confirm the proliferation measurements.

After 144 h, cell monolayers were rinsed and incubated for 3 h at 37° C., with fresh medium containing soluble MTT. Formazan crystals produced by viable cells were then dissolved in dimethylsulfoxide (DMSO), and the intensity of the resulting blue color was measured at 540 nm using ThermoMax microplate reader (Molecular Devices). Data were analyzed using SoftMax software. Results are expressed as inhibition of proliferation compared to cell growth in the control samples. 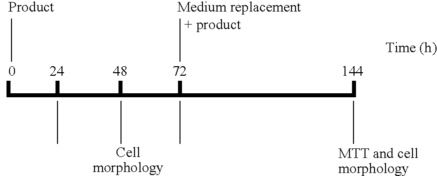
Results and Conclusions

Table 5 summarizes the effect of tomato-derived anti-proliferative composition on the proliferation of normal human dermal fibroblasts. Cell observation represents the apparent relative cell count (%) after various incubation times (proliferation index compared to the proliferation index of the control after the same incubation time).

TABLE 5
Proliferation of normal human dermal fibroblasts population after
treatment with tomato derived anti-proliferative composition

| | | | | | Proliferation | |
|---|---|---|---|---|---|---|
| Dilution | | | | | index | Inhibition of |
| (Tomato-derived | | | | | (% Of | proliferation |
| anti-proliferative | Cell observations | | | | control) | (%) |
| composition) | 24 h | 48 h | 72 h | 144 h | After 144 h; n = 6 | |
| 1/10 | 75%* | 50%* | 25* | 10%* | 25 | 75 |
| 1/20 | 90-75%^{#} | 75-50%^{#} | 50%^{#} | 50%^{#} | 50 | 50 |
| 1/40 | 100% | 75%^{#} | 75%^{#} | 75%^{#} | 81 | 19 |
| 1/80 | 100% | 100% | 100% | 90%^{#} | 102 | 0 |
| 1/160 | 100% | 100% | 100% | 100% | 109 | 0 |

*Cells were blocked, no division seemed to occur; the cell density was apparently the same as that at the beginning of the experiment.
^{#}Cells were not totally blocked; the cell density was apparently higher compared to that at the beginning of the experiment.

Using the above-described in vitro model and protocol, the tomato-derived anti-proliferative composition showed a cytostatic effect for dilution between 1/10 and 1/40.

Test Compound: Pitaya-Derived Anti-Proliferative Composition as Stock Solution

Materials and Methods

Biological model

| | |
|---|---|
| Type: | Pool of normal human dermal fibroblast (pool No. |
| | PF2NHDF 9^{th} passage). |
| Culture medium: | DMEM (Invitrogen 21969035); Glutamine 2 mM |
| | (Invitrogen 25030024); Penicillin 50 UI/ml; |
| | Streptomycin 50 μg/ml (Invitrogen 15070063); Fetal |
| | calf serum 10% (Invitrogen 102700981) |

Dilutions

The source pitaya derived anti-proliferative composition was diluted in sterile culture medium. Concentrations assayed were 1/10; 1/20; 1/40; 1/80; and 1/160 dilution of the raw pitaya-derived anti-proliferative composition stock solution.

Assay

The protocol used in this study was the same used for tomato-derived anti-proliferative composition as described herein above.

Results and Conclusions

In this in vitro study, the pitaya-derived anti-proliferative composition decreased the MTT labeling compared to the control cultures at a dilution of 1/10 of the stock solution. Lower concentrations had no significant effect (Table 6). The pitaya-derived anti-proliferative composition was not cytotoxic, as the cells continued to grow, only at a lower rate compared to cell grown without the composition; Thus, the composition was shown to have a cytostatic effect.

TABLE 6
Proliferation of normal human dermal fibroblasts population after
treatment with pitaya-derived anti-proliferative composition

| Dilution | | | | | | |
|---|---|---|---|---|---|---|
| (Pitaya- | | | Proliferation | | | |
| derived anti- | | | index | | Inhibition of | |
| proliferative | Cell density | | (% Of control) | | proliferation (%) | |
| composition) | 72 h | 144 h | 72 h | 144 h | 72 h | 144 h |
| 1/10 | 90% | 75% | 83 | 58 | 17 | 42 |
| 1/20 | 100% | 100% | 101 | 88 | 0 | 12 |
| 1/40 | 100% | 100% | 106 | 101 | 0 | 0 |
| 1/80 | 100% | 100% | 104 | 104 | 0 | 0 |
| 1/160 | 100% | 100% | 105 | 101 | 0 | 0 |

### Example 4

### Comparison of the Anti-Proliferative Activity of Non-Autoclaved and Autoclaved Tomato-Derived Compositions

The extraction processes of the composition of the present invention include heating the extract to at least 65° C., and thus the extracted anti-proliferative agents are heat stable. To further examine the heat-stability of the agents, the anti-proliferative effect of autoclaved tomato-derived compositions on human fibroblasts was assayed. Material and methods are as described in Example 4 herein above. The assay was conducted with tomato-derived composition (designated herein IBR-Tom) vs. autoclaved composition (autoclaved IBR-Tom). Material and methods are as described in Example 3 hereinabove.

Results and Conclusions

Table 7 summarizes the effect of IBR-Tom and IBR-Tom autoclaved on the viability and proliferation of the fibroblast cells.

TABLE 7
Viability and proliferation of normal human dermal fibroblasts
population after treatment with IBR-Tom or IBR-Tom Autoclaved

| | | Cell observations | | | | Inhibition of |
|---|---|---|---|---|---|---|
| Treatment | Concentration | 24 h | 48 h | 72 h | 144 h | proliferation (%) |
| IBR-Tom | 1/10 | 75%* | 50-75%″ | 50-75%″ | 50%″ | 39 |
| | 1/20 | 100% | 75%″ | 75%″ | 75%″ | 17 |
| | 1/40 | 100% | 100% | 100% | 100% | 2 |
| | 1/80 | 100% | 100% | 100% | 100% | 2 |
| | 1/160 | 100% | 100% | 100% | 100% | 0 |
| IBR-Tom | 1/10 | 75%* | 50%″ | 25%″ | 10%* | 75 |
| Autoclaved | | | | | | |
| | 1/20 | 75-90%″ | 50-75%″ | 50%″ | 50%″ | 50 |
| | 1/40 | 100% | 100% | 100% | 100% | 19 |
| | 1/80 | 100% | 100% | 100% | 100% | 0 |
| | 1/160 | 100% | 100% | 100% | 100% | 0 |

*Cells were blocked, no division seemed to occur; the cell density was apparently the same as that at the beginning of the experiment.
″Cells were not totally blocked; the cell density was apparently higher compared to that at the beginning of the experiment.

Both MTT and microscopic evaluation showed that “IBR-Tom autoclaved” was cytostatic at a lower dose than “IBR-Tom”. “IBR-Tom” was cytostatic at the dilution 1/20 and “IBR-Tom autoclaved” at 1/40.

With this in vitro model and this protocol IBR-Tom showed a cytostatic effect for concentration between 1/10 and 1/20 and IBR-Tom autoclaved between 1/10 and 1/40.

### Example 5

### Toxicity Potential of Tomato Derived Anti-Proliferative Composition

Cytotoxicity

Cytotoxicity was assessed by an agarose diffusion test, in which the test material is applied to the surface of agarose gel, wherein the agarose gel is in contact with cells. Cytotoxic test material causes cell lysis. Live cells incorporate MTT and transform it to formazan as described herein above; cytotoxicity potential is given according to the mean area of non-stained cells, i.e., lysed cells, by the following scale:

| Mean diameter of lysis in cm | Classification |
|---|---|
| <2.0 | Weak cytotoxicity |
| 2.0-3.0 | Moderate cytotoxicity |
| ≧3.0 | Significant cytotoxicity |

Two independent tests were performed in duplicate (total of 4 Petri dishes). Cultured cell were trypsinized and counted. 2×10⁶ cells in 4 ml of DMEM medium were seeded in each 50 mm diameter Petri dish. The dishes were incubated for 24 h±1 h at 37° C., 5% CO₂, before they were covered with 4% agarose gel, prepared with complete DMEM medium. The test compound (tomato derived anti-proliferative composition, TC) was applied on top of a 6 mm disc of filter paper that was placed in the center of the agarose gel surface.

After 23 h-25 h of contact at 37° C. and 5% CO₂ the filter paper with the test compound and the agarose gel were gently removed. The cells were rinsed carefully with PBS by a pipette. The liquid was then removed, and 2 ml solution of MTT at 0.5 mg/ml, prepared extemporaneously from a source solution of 5 mg/ml, was added to each dish. The dishes were then incubated for 0.5-1.5 h at 37° C. and 5% CO₂. After removal of the excess dye, living cells were colored while lysed cells appeared as an uncolored zone. Each dish was placed on a light surface and the largest and the smallest diameters of the lysis area, estimated visually, were measured by a measuring ruler (mm) on a graph paper, and the mean diameter was calculated.

The value of the diameter of cell lysis taken into account for the determination of cytotoxicity corresponded to the arithmetical mean of the mean diameter defined for the 2 dishes of each test (MD). Pure complete DMEM served as a negative control (no lysis should occur). 3% SDS served as a positive control (cells are lysed due to the presence of SDS).

The assay results are summarized in table 8 below.

TABLE 8
Cytotoxicity of tomato-derived anti-proliferative composition measured
by agarose diffusion test

| | Largest diameter | | | | Smallest diameter | | | | Mean | | | | MD | |
|---|---|---|---|---|---|---|---|---|---|---|---|---|---|---|
| Dish No. | 1 | 2 | 3 | 4 | 1 | 2 | 3 | 4 | 1 | 2 | 3 | 4 | 1 + 2 | 3 + 4 |
| DMEM | 0.0 | 0.0 | 0.0 | 0.0 | 0.0 | 0.0 | 0.0 | 0.0 | 0.0 | 0.0 | 0.0 | 0.0 | 0.0 | 0.0 |
| 3% SDS | 2.5 | 2.6 | 2.6 | 2.5 | 2.4 | 2.5 | 2.5 | 2.4 | 2.5 | 2.6 | 2.6 | 2.5 | 2.6 | 2.6 |
| TC | 0.0 | 0.0 | 0.0 | 0.0 | 0.0 | 0.0 | 0.0 | 0.0 | 0.0 | 0.0 | 0.0 | 0.0 | 0.0 | 0.0 |

As demonstrated in table 8, the tomato-derived anti-proliferative composition caused no visual cell lysis, and was therefore characterized as having weak cytotoxicity.

Compatibility of Human Skin to the Tomato Derived Anti-Proliferative Composition

The skin compatibility to the tomato-derived composition of the present invention was examined after single application to the skin of volunteers under exaggerated experimental conditions. As used herein, “exaggerated experimental conditions” refers to single application of the composition (20 μl) to the skin, under patch, for 48 h (“Patch test”).

After 48 h the patch was removed. After additional 15 minutes, the skin area that was under the patch was examined visually by a qualified person. Estimation was made in comparison to a “negative” control: patch with distilled water, non-irritant, which was applied in parallel and under the same conditions as the test product.

Nine volunteers withstanding the inclusive criteria detailed below participated in the experiment.

Inclusive criteria:
Age: 18 to 70 years old,
Gender: female and/or male,
Phototype (Fitzpatrick): I to V,
Free of all dermatological lesions on the site studied

None of the volunteers reacted to the composition applied to the skin, and therefore the mean daily irritation score, according to the present study, is zero. The tomato derived anti-proliferative composition is therefore characterized as not irritant regarding its primary cutaneous tolerance, and thus as having good compatibility to human skin.

The cytotoxicity and irritation potential of the tomato-derived anti-proliferative composition was further assessed by the natural red release assay. This assay is based on measuring the release of pre-incubated natural red dye (3-amino-7dimethylamino-2-methylphenazine hydrochloride) by normal epithelial cell cultures following exposure to a test material. In the presence of cytotoxic test materials, which cause damage to the cell membrane, an increase in the release of the natural red dye is observed. Employing this test with cultures of fibroblast isolated from rabbit cornea, showed that the tomato-derived anti-proliferative composition a negligible cytotoxicity.

### Example 6

### Toxicity Potential of Narcissus Derived Anti-Proliferative Composition

Mutagenesis Potential

The narcissus-derived anti-proliferative composition was assayed for its potential to cause mutation by the Ames test (Ames B. N., McCann, J., and Yamasaki E., Mutation Research, 31:347-364 (1975). Briefly, the test is based on the ability of a substance to reverse a mutation in a strain of Salmonella typhimurium such that the bacteria are able to grow on a medium lacking histidine. The Narcissus derived anti-proliferative composition did not induce any mutagenic effect up to a dose of 5,000 μg/plate.

Cytotoxicity

Cytotoxicity of the narcissus derived anti-proliferative composition was assessed by an agarose diffusion test as described for the tomato-derived composition herein above. The Narcissus derived composition was also characterized as having weak cytotoxicity.

Cutaneous Tolerance

Cutaneous tolerance of the narcissus derived composition ((5%) of 0.2 gr/ml extract) in cosmetic cream, after repeated application to the skin was assessed by EVIC-CEBA, Bordeaux, France. The product was found very well tolerated by the skin.

### Example 7

### Toxicity Potential of Pitaya-Derived Anti-Proliferative Composition

The toxicity potential of the pitaya-derived anti-proliferative composition was assesses employing the natural red release assay and the “patch test” described herein above. The natural red assay results demonstrated that the pitaya-derived composition is also defined as having a negligible cytotoxicity. Ten healthy adult volunteers participated in the patch test. After single application of 20 μl of the composition, under occlusive patch and during 48 hours, no irritation signs could be detected (mean daily irritation score=0). Therefore, the pitaya-derived anti-proliferative composition is considered as not irritant regarding its primary cutaneous tolerance.

### Example 8

### Cosmetic and Pharmaceutical Compositions

The cosmetic and pharmaceutical compositions are illustrated by the following formulation examples. Anti-proliferative composition refer to the plant derived anti-proliferative compositions according to the present invention.

Topical Application

| | |
|---|---|
| Ingredient | Amount (g) |

A. Balm

| | |
|---|---|
| Ozokerite | 20 |
| White Vaseline | 14.0 |
| Isopropyl palmitate | 9.0 |
| Perfume | 1.0 |
| Antioxidants | 0.3 |
| Preserving agent | 0.2 |
| Anti-proliferative composition | 0.02 |
| Liquid paraffin | sqf 100.0 |

B. Balm

| | |
|---|---|
| Ozokerite | 19.0 |
| White Vaseline | 15.0 |
| Anti-proliferative composition | 1.0 |
| Antioxidant | 0.3 |
| Preserving agent | 0.2 |
| Liquid purcellin oil | sqf 100.0 |

C. Emulsified gel of O/W type

| | |
|---|---|
| Ethyl alcohol | 15.0 |
| Purcellin oil | 7.0 |
| Anti-proliferative composition | 3.0 |
| Volatile silicone oil | 3.0 |
| Carbopol ® 981 (marketed by Goodrich) | 0.6 |
| Perfume | 0.4 |
| Preservative agent | 0.3 |
| Triethanolamine | 0.2 |
| Demineralized water | sqf 100.0 |

D. Aqueous-alcoholic gel

| | |
|---|---|
| 95% Ethanol | 60.0 |
| Glycerol | 3.0 |
| Propylene glycol | 2.0 |
| Carbopol ® 981 (marketed by Goodrich) | 1.0 |
| Triethanolamine | 1.0 |
| Anti-proliferative composition | 0.5 |
| Perfume | 0.4 |
| Demineralized water | sqf 100.0 |

E. Anhydrous gel

| | |
|---|---|
| Propylene glycol | 25.0 |
| Polyethylene glycol | 12.0 |
| Hydroxyethyl cellulose | 0.8 |
| Anti-proliferative composition | 0.0001 |
| Absolute ethanol | sqf 100 |

F. Emulsion of O/W type

| | |
|---|---|
| Volatile silicone oil | 10.0 |
| Anti-proliferative agent | 10.0 |
| Liquid paraffin | 6.0 |
| Arlacel ® 165 (marketed by Atlas) | 6.0 |
| Liquid lanolin | 3.0 |
| Stearic acid | 2.5 |
| Tween ® 60 (marketed by Atlas) | 2.0 |
| Cetyl alcohol | 1.2. |
| Preserving agent | 0.3 |
| Antioxidants | 0.3 |
| Triethanolamine | 0.1 |
| Demineralized water | sqf 100 |

G. Emulsion of O/W type

| | |
|---|---|
| Cetyl alcohol | 3.0 |
| Stearic acid | 3.0 |
| Glycerol | 3.0 |
| PEG 400 | 3.0 |
| Propylene glycol | 2.0 |
| Corn oil | 2.0 |
| Isopropyl myristate | 1.0 |
| Perfume | 0.5 |
| Preserving agent | 0.3 |
| Carbopol ® 981 (marketed by Goodrich) | 0.2 |
| Anti-proliferative composition | 0.1 |
| Demineralized water | sqf 100.0 |

H. Clear gel

| | |
|---|---|
| Ethyl alcohol | 30.0 |
| Oxyethylenated nonylphenol | 5.0 |
| Glycerin | 3.0 |
| Carbopol ® 981 (marketed by Goodrich) | 1.0 |
| Triethanolamine | 0.3 |
| Perfume | 0.3 |
| Preserving agent | 0.3 |
| Anti-proliferative composition | 0.005 |
| Demineralized water | sqf 100.0 |

I. Cream containing liposomes

| | |
|---|---|
| Sunflower oil | 35.0 |
| Cetyl alcohol | 4.0 |
| B-sitosterol | 4.0 |
| Perfume | 0.6 |
| Dicetyl phosphate | 0.5 |
| Preserving agent | 0.3 |
| Carbopol ® 981 (marketed by Goodrich) | 0.2 |
| Triethanolamine | 0.2 |
| Sphingosine | 0.05 |
| Anti-proliferative composition | 0.0002 |
| Demineralized water | sqf 100.0 |

J. Per os composition

| Ingredient | Amount (mg) |
|---|---|
| Anti-proliferative composition | 20.0 |
| Talc | 5.0 |
| Aerosil 200 | 5.0 |
| Stearate de Zn | 5.0 |
| Lactose | sqf 400.0 |
| Ingredient | Amount (g) |

K. Liquid for Iontophoresis

| | |
|---|---|
| Anti-proliferative composition | 3.0 |
| Preserving agent | 0.15 |
| Benzoate de sodium | 0.02 |
| Water | sqf 100.0 |

L. Emulsion W/O

| | |
|---|---|
| Protegin | 19.0 |
| Vaseline oil | 8.0 |
| Glycerin | 3.0 |
| Anti-proliferative composition | 2.0 |
| Perfume | 0.8 |
| Sulfate de Mg | 0.5 |
| Preserving agent | 0.2 |
| Water | sqf 100.0 |

### Example 9

### Agricultural Application of Narcissus-Derived Anti-Proliferative Composition

As described herein above, the narcissus derived anti-proliferative composition of the present invention inhibits root growth after the onset of germination. Accordingly, the composition was examined as an inhibitor of root development. Such an application would be very useful in germplasm preservation and propagation by tissue culture, as it could significantly reduce the need for sub-culturing and thus reducing labor and media cost, and/or reduce the need for mass multiplication of shoots in micropropagation.

Narcissus derived anti-proliferative composition was examined for its activity as an inhibitor of root development by several experiments.

Experiment I

This experiment examined the efficacy of the narcissus derived anti-proliferative composition as a root inhibitor of impatiens (Impatien walleriana) in cell culture. Impatiens is an important commercial floricultural crop. Under existing protocols, shoot regeneration of impatiens in tissue culture has been difficult with common explants, such as leaf sections or cotyledons, because the explants tend to form roots exclusively and abundantly. A successful root inhibitor may therefore improve regeneration protocols by shifting the balance in the direction of shoot formation.

Impatiens walleriana (accent red) seeds were surfaced sterilized by dipping in 85% EtOH for 5 sec, followed by incubation with 30% bleach for 17 minutes. The seeds were then rinsed 4 times with sterile ddH₂O, 5 min per rinse, with 100 ml rinse water in 250 ml beaker.

Seeds were then germinated under aseptic conditions on 10% strength MS Basal media for 10-15 days. Explants having cotyledons and hypocotyls, were then excised and placed on modified MS Basal media amended with 10 μM BA and 0.1 μM IAA, and 0, 1, 2, 5, or 10% narcissus derived anti-proliferative composition. Explants where then placed in an incubator (16 h light 24° C. 8 h Dark 18° C.) for 14 or 15 days.

The narcissus derived anti-proliferative composition was a very powerful rooting inhibitor of impatiens explants in tissue culture. Medium containing 2% of the composition was very effective with only about 3% of the explants showing any sign of root development. At a concentration of one percent, only 27% of explants showed any signs of root development compared to 100% in controls. These results indicate that the narcissus derived anti-proliferative composition is an effective inhibitor of rooting of Impatiens in culture.

Experiment II

This experiment further examined the effect of narcissus derived anti-proliferative composition on rooting of shoot cuttings of tomato (Lycopersicon esculentum) and coleus (Coleus blumei) plants. Both plants are known for their ability to easily produce roots from cuttings when incubated in water.

Mother plants were grown outdoor and cuttings were incubated in either 0, 1, 5, or 10% narcissus derived anti-proliferative composition in 125 ml flasks filled with about 100 ml solution. Stems of the cuttings were submerged 3 to 5 cm in the solution. Flasks were refilled with stock solution to make up for evaporative losses during the experiment. Plants were incubated on a laboratory bench where they received full sunlight for approximately 3 hours per day and artificial light for an additional 6 hours per day. The temperature was maintained at about 20-25° C. The experiment continued for 25 days.

The stem tissue in direct contact with the narcissus derived composition exhibited significant root inhibition (table 9). In several cases, roots developed normally just above the water line demonstrating that the composition was an effective root inhibitor when plants were in direct contact with the solution.

TABLE 9
Rooting of Tomato cuttings after 21 Days after Exposure to
narcissus derived anti-proliferative composition

| Concentration of | | |
|---|---|---|
| the narcissus | Percent of Plants | |
| derived | that developed | |
| composition | roots | Observations |
| 0 | 100% | Root in 2 to 3 days |
| 1 | 21% | Partial inhibition of |
| | | root development |
| 5 | 2% | Inhibition of root |
| | | development |

Coleus is another prolific root producer. A pilot study using lower concentrations of the narcissus derived anti-proliferative composition compared to the concentrations described above for tomato was conducted. At 0.01% of the composition rooting was delayed, and after initiation roots grew slowly. At 0.025%, coleus root formation was inhibited when the solution comprising narcissus derived anti-proliferative composition was in direct contact with the roots. At 0.05% the inhibition was more pronounced and only few roots developed. Direct contact with the solution was required to obtain inhibition of root formation and growth.

In summary, the narcissus derived anti-proliferative composition was shown to be an effective inhibitor of plant root development and growth, when in constant contact with the plant tissue. The most effective inhibitory concentrations seemed to be in a range from approximately 0.5% to 5% v/v. The effective concentration varies among plant species, the age of plant material, and the application e.g. tissue culture versus rooted cuttings in solution.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. An anti-proliferative composition comprising at least one plant-derived anti-proliferative agent, wherein the anti-proliferative agent:
a. is a water soluble, small organic molecule;
b. induces or maintains dormancy in at least one organ of the plant;
c. inhibits exogenic cell proliferation; and
d. its inhibitory activity is reversible.

2. The composition according to claim 1, wherein the anti-proliferative agent has a molecular weight of less than about 5,000 Dalton.

3. The composition according to claim 2, wherein the anti-proliferative agent is heat stable.

4. The composition according to claim 1, wherein the inhibition of exogenic cell proliferation is measured by exposing a tissue or cell culture to different concentrations of said composition and measuring proliferation rate of the tissue or cell culture, wherein a decrease in the proliferation rate as compared to proliferation rate of said tissue or cell culture incubated without the composition is observed.

5. The composition according to claim 4, wherein the reduction in the proliferation rate is at least about 20%.

6. The composition according to claim 1, wherein the anti-proliferative agent is obtained from a dormant plant organ selected from the group consisting of a seed, an apical vegetative bud, a lateral vegetative bud, a floral bud, a bulb, a corm, and a tuber.

7. The composition according to claim 1, wherein the anti-proliferative agent is obtained from a plant tissue surrounding a dormant organ.

8. The composition according to claim 7, wherein the dormant organ is a seed and the tissue surrounding the dormant organ is a fruit or a part thereof.

9. The composition according to claim 8, wherein the fruit is a fleshy fruit selected from the group consisting of grape, kiwi, grapefruit, pitaya and tomato.

10. The composition according to claim 1, formulated in a form selected from the group consisting of a solution, a suspension, an emulsion and a dry soluble lyophilized powder.

11. The composition according to claim 10, further comprising at least one additional ingredient selected from the group consisting of a preservative, an antioxidant or a combination thereof.

12. A cosmetic composition comprising as an active ingredient an anti-proliferative composition according to claim 1, further comprising a cosmetically acceptable diluent or carrier.

13. The cosmetic composition according to claim 12, wherein the anti-proliferative composition comprises at least one anti-proliferative agent having a molecular weight of less than about 5,000 Dalton.

14. The cosmetic composition according to claim 13, wherein the anti-proliferative agent is heat stable.

15. The cosmetic composition according to claim 12, further comprising at least one agent selected from the group consisting of a preservative, a thickener, a dispersing agent, an emulsifier, a colorant, a perfume or any combination thereof.

16. The cosmetic composition according to claim 12, further comprising at least one active ingredient selected from the group consisting of an anti-oxidant, an anti-inflammatory agent, a moisturizer, a vitamin, a carotenoid, a UV absorbing agent a UV protecting agent or any combination thereof.

17. The cosmetic composition according to claim 12, formulated for topical application, in a form selected from the group consisting of balm, cream, emulsion, gel, hydrophilic oil, liposome, lotion, mousse, capsule, ointment, suspension, solution, salve, and an impregnated dressing.

18. The cosmetic composition according to claim 17, wherein the solution further comprises at least one solvent selected from the group consisting of water, buffered aqueous solution, ethyl alcohol, isopropyl alcohol, propylene glycol, butylene glycol, polyethylene glycol, glycerin, glycoforol, ethyl lactate, methyl lactate, N-methylpyrrolidone, ethoxylated tocopherol, or any combination thereof.

19. The cosmetic composition according to claim 17, wherein the gel is in a form selected from an aqueous-alcoholic gel and a clear gel.

20. A pharmaceutical composition comprising as an active ingredient a therapeutically effective amount of anti-proliferative composition according to claim 1, further comprising a pharmaceutically acceptable diluent, excipient or carrier.

21. The pharmaceutical composition according to claim 20, wherein the anti-proliferative composition comprises at least one anti-proliferative agent having a molecular weight of less than about 5,000 Dalton.

22. The pharmaceutical composition according to claim 13, wherein the anti-proliferative agent is heat stable.

23. The pharmaceutical composition according to claim 20, formulated for topical application, in a form selected from the group consisting of a balm, cream, emulsion, gel, hydrophilic oil, liposomes, lotion, mousse, capsule, ointment, suspension, solution, salve, or any impregnated dressing.

24. The pharmaceutical composition according to claim 23, wherein the solution further comprises at least one solvent selected from the group consisting of water, buffered aqueous solution, ethyl alcohol, isopropyl alcohol, propylene glycol, butylene glycol, polyethylene glycol, glycerin, glycoforol, ethyl lactate, methyl lactate, N-methylpyrrolidone, ethoxylated tocopherol, dimethylsulfoxide (DMSO), tetrahydrofuran (THF), or any combination thereof.

25. The pharmaceutical composition according to claim 23, wherein the gel is in a form selected from an aqueous-alcoholic gel and a clear gel.

26. The pharmaceutical composition according to claim 20 formulated for oral application, in a form selected from the group consisting of a capsule, dragee, pill, tablet, gel, liquid, slurry, suspension and syrup.

27. An agricultural composition comprising as an active ingredient an agricultural effective amount of anti-proliferative composition according to claim 1, further comprising agriculturally acceptable diluent or carrier.

28. The agricultural composition according to claim 27, wherein the anti-proliferative composition comprises at least one anti-proliferative agent having a molecular weight of less than about 5,000 Dalton.

29. The agricultural composition according to claim 28, wherein the anti-proliferative agent is heat stable.

30. The composition according to claim 27, further comprising at least one active ingredient selected from the group consisting of a pesticide, a fungicide, an anti-biotic agent, a herbicide a nutrient or any combination thereof.

31. A method for at least caring for, making up or protecting the human skin, comprising applying to the skin a cosmetic composition according to claim 12.

32. The method according to claim 31, for at least one of reducing aging signs, reducing wrinkles, promoting skin firmness, reducing skin sensitivity, reducing skin irritability or any combination thereof.

33. The method according to claim 31, wherein the skin is protected from external aggressions.

34. The method according to claim 33, wherein the external aggression is selected from the group consisting of radiation, sun radiation, ozone, acid rain, extreme temperature, transport pollutants, industry pollutants, cleaning materials, drugs, toxins or any combination thereof.

35. A method for slowing cell proliferation comprising applying to a subject in need thereof a cosmetic composition according to claim 12 in an amount effective in reducing cell proliferation.

36. The method according to claim 35, wherein slowing cell proliferation is beneficial for at least one phenomenon selected from the group consisting of reducing undesired hair growth, reducing nail growth, reducing acne, obtaining better scar formation, reducing alopecia, reducing skin sebum, enhancing skin whitening, extending the duration of a tan or any combination thereof.

37. A method for treating undesired or deleterious cell proliferation comprising administering to a subject in need thereof a therapeutically effective amount of a pharmaceutical composition according to claim 20.

38. The method according to claim 37, wherein the undesired or deleterious cell proliferation is associated with a disease or disorder selected from the group consisting of malignant cell proliferation, psoriasis, seborrehic keratosis, fibrosis, restenosis, wart infection and papilloma infection.

39. The method according to claim 38, for treatment of malignant cell proliferation.

40. The method according to claim 39, for the treatment of carcinoma.

41. The method according to claim 39, for the treatment of melanoma.

42. The method according to claim 39, for the treatment of hyperproliferative mammalian cells with drug-resistant phenotypes.

43. The method according to claim 39, wherein the treatment is applied in combination with at least one additional anti cancer treatment.

44. The method according to claim 43, wherein the additional anti cancer treatment is selected from the group consisting of radiation therapy, chemotherapy, immunotherapy, hormonal therapy and genetic therapy.

45. A method for slowing cell proliferation in a plant tissue, the method comprising applying to the plant tissue an agricultural composition according to claim 27, in an amount effective in reducing plant cell proliferation.

46. The method according to claim 25, for at least one of controlling root elongation, reducing the water requirement of a plant and prolonging the storage period of a plant part.

47. The method according to claim 46, wherein the plant part is selected from the group consisting of cutting, cut flower, fruit and seed.
